(19) United States
(12) Patent Application Publication
(10) Pub. No.: US 2024/0212812 A1
(43) Pub. Date: Jun. 27, 2024

(54) INTELLIGENT MEDICAL REPORT GENERATION

(71) Applicant: Stryker Corporation, Kalamazoo, MI (US)

(72) Inventors: Tejas Haritsa VIDYASHANKAR KERESANTHE, Bengaluru (IN); Bhavya Nishitkumar AJANI, Bengaluru (IN); Rushit Dharmendrabhai BHADANIYA, Gurugram (IN); Joel M. ERNST, Dallas, TX (US)

(73) Assignee: Stryker Corporation, Kalamazoo, MI (US)

(21) Appl. No.: 18/391,616

(22) Filed: Dec. 20, 2023

Related U.S. Application Data
(60) Provisional application No. 63/477,372, filed on Dec. 27, 2022.

(51) Int. Cl. G16H 15/00; G16H 30/20

(52) U.S. Cl. CPC G16H 15/00; G16H 30/20

(57) ABSTRACT

Described herein are systems, methods, and programming for generating medical reports describing medical procedures. The draft medical report may include auto-generated content describing a medical procedure, where the auto-generated content may include one or more auto-generated images selected based on medical report criteria. The draft medical report may be generated and a user selection of at least one of the auto-generated images may be received. The draft medical report may be updated based on the user selection.

### CROSS-REFERENCE TO RELATED APPLICATIONS

This application claims the benefit of U.S. Provisional Application No. 63/477,372, filed Dec. 27, 2022, the entire contents of which are hereby incorporated by reference herein.

### FIELD

The present application generally relates to generating medical reports, and more specifically to interfaces for interacting with and updating medical reports.

### BACKGROUND

Medical reports may include images, videos, and/or text that describe various aspects of a medical procedure. Traditionally, a user, such as a medical professional, may capture one or more images during a medical procedure. These images may be included in the medical report. However, the manual capture of the images may require the user to later recall details about each image for inclusion in the medical report. The alternative would be to have the user pause the medical procedure after each image is captured, provide written or spoken notes to go along with the image, and then resume the medical procedure. Additionally, if graphics are to be added, such as to reference anatomical structures, the user may have to manually input the graphics after the medical procedure has been completed.

Therefore, it would be beneficial to have systems, methods, and programming that facilitate automatic and intelligent generation of medical reports including images captured during a procedure.

### SUMMARY

Described are systems, methods, and programming for automating a medical report generation process. A medical report may include content, such as images, video, text, and/or audio, that describes particular medical events from a medical procedure. The medical procedure may include a surgical procedure (e.g., minimally invasive surgical (MIS) procedures, non-invasive procedures, invasive procedures, etc.). This content has been traditionally captured manually by a user (e.g., a surgeon, medical professional, imaging specialist, etc.). For example, a surgeon may capture an image from an intraoperative video of a medical procedure when the intraoperative video depicts a particular medical event. Following the medical procedure, these images may be reviewed by the surgeon to select which images to include in the medical report. However, this postoperative selection process relies on the user to recall pertinent details related to the particular medical events depicted by the captured images. This reliance on the user to recall details related to various earlier-captured images can lead to crucial information being forgotten and not included in the medical report. In addition to or instead of adding information after a medical procedure, the surgeon may pause the surgery to input details related to medical events. However, pausing a medical procedure (e.g., a surgical procedure) may increase an amount of time it takes to perform the medical procedure, deviate the user's focus from patient care (e.g., a surgeon, medical staff, etc.), and/or otherwise impede the medical procedure's workflow.

The aforementioned medical events may comprise key moments that transpire during the medical procedure, which can be identified based on prior performances of the medical procedure. For example, prior performances of the medical procedure may indicate the moments at which an image was captured to document given key moments during the medical procedure. A machine learning model may be trained to identify features present within the captured images and related to key moments in the medical procedure. Thus, images captured during a performance of the medical procedure may be input to the trained machine learning model to detect whether any of the images depict features related to key moments in the medical procedure. If it is determined that one or more images depict these features, the trained machine learning model may extract and store those images for later inclusion in a draft medical report describing the performance of the medical procedure. After the medical procedure has completed, a draft medical report may be generated including at least some of the images depicting the documented key moments of the surgical procedure.

In addition to capturing images of key moments determined from prior performances of the medical procedure, the machine learning model may be trained to detect medical events that may be beneficial to include within the medical report. For example, if a particular abnormal action is detected during the medical procedure, the trained machine learning model may detect the abnormal action and capture one or more images of the abnormal action. As another example, the trained machine learning model may detect the presence of an object within a captured image that may be of importance to the medical procedure and may select that image of the object for inclusion within the draft medical report.

The images depicting the medical events may be presented to the user in addition to the draft medical report. The user may select one or more of the images depicting the medical events to include in the draft medical report. The draft medical report can be updated to include selected image(s) as well as any other additional information as identified by the user to be included within the medical report. In this way, not only is the draft medical report automatically created based on user preference, content describing important events that occurred during the medical procedure can be automatically identified and provided to the user as optional content to add to the draft medical report.

According to some examples, a method includes generating a draft medical report comprising auto-generated content describing a medical procedure, wherein the auto-generated content comprises one or more auto-generated images that have been selected based on medical report criteria; displaying the draft medical report comprising the one or more auto-generated images; receiving a user selection of at least one of the one or more auto-generated images; and updating the draft medical report based on the user selection of the at least one of the one or more auto-generated images. The medical report criteria may e.g. comprise one or more of a user identifier (ID), procedure preferences, model preferences, report preferences, other preferences of a user, other information relating to the user, etc.

In any of the examples, the method can further include: selecting a medical profile of a user associated with the medical procedure, the medical profile comprising the medical report criteria. The medical report criteria may include preferences of the user provided in the medical profile of the user.

In any of the examples, the method can further include: determining the medical report criteria based on a type of the medical procedure. A first type of medical procedure may be associated with first medical report criteria, while a second type of medical procedure may be associated with second medical report criteria.

In any of the examples, the method can further include: identifying one or more time windows associated with the medical procedure; and capturing an image during at least one of the one or more time windows, wherein the one or more auto-generated images comprise the captured image.

In any of the examples, the method can further include: obtaining a medical profile of a user associated with the medical procedure; and identifying one or more medical report preferences of the user based on the medical profile, the one or more medical report preferences indicating time windows of the medical procedure during which the user prefers to capture images, wherein the one or more auto-generated images comprise at least some of the captured images.

In any of the examples, the method can further include: obtaining auto-generated text describing each of the one or more auto-generated images, wherein the auto-generated content comprises the auto-generated text, and wherein the draft medical report comprises the one or more auto-generated images and the auto-generated text corresponding to each of the one or more auto-generated images.

In any of the examples, the method can further include: generating graphics associated with the one or more auto-generated images.

In any of the examples, the method can further include: displaying the updated draft medical report comprising at least some of the auto-generated content and the at least one of the one or more auto-generated images.

In any of the examples, updating the draft medical report can include: adding the at least one of the one or more auto-generated images to the draft medical report to obtain the updated draft medical report.

In any of the examples, the method can further include: determining one or more medical events associated with the medical procedure based on prior performances of the medical procedure; and generating a medical profile of a user associated with the medical procedure, the medical profile storing data indicating the one or more medical events.

In any of the examples, the method can further include: detecting, within a video of the medical procedure, at least some of the one or more medical events; and selecting one or more images depicting the at least some of the one or more medical events, wherein the auto-generated content comprises at least some of the one or more captured images.

In any of the examples, the method can further include: training a machine learning model to identify one or more image descriptors associated with phases of the medical procedure; and capturing, from video of the medical procedure, one or more images corresponding to the phases of the medical procedure, the auto-generated content comprising at least some of the one or more captured images.

In any of the examples, the one or more image descriptors can comprise at least one of objects, environmental factors, or contextual information associated with the phases of the medical procedure.

In any of the examples, the method can further include: generating training data comprising images that were captured during prior performances of the medical procedure for training the machine learning model; and storing at least one of the trained machine learning model or the training data in association with a medical profile of a user that performed the medical procedure.

In any of the examples, the method can further include: detecting, within video of the medical procedure, using the trained machine learning model, at least one of the one or more image descriptors; and selecting one or more images from the video of the medical procedure depicting the at least one of the one or more image descriptors, the auto-generated content comprising at least some of the one or more selected images.

In any of the examples, the at least one of the objects can include an anatomical structure.

In any of the examples, the method can further include: determining time windows associated with phases of the medical procedure; and detecting an image captured at a time different than the time windows, wherein the one or more auto-generated images comprise the detected image.

In any of the examples, the method can further include: associating audio captured during the medical procedure with an image captured during a time window associated with a phase of the medical procedure.

In any of the examples, the method can further include: generating user-provided text representing the audio; merging the user-provided text with auto-generated text associated with the captured image, wherein the draft medical report comprises the captured image and the merged text.

In any of the examples, generating the draft medical report can comprises: determining, based on a medical profile of a user associated with the medical procedure, one or more medical report preferences of the user; and creating the draft medical report based on the one or more medical report preferences.

In any of the examples, the method can further include: updating the one or more medical report preferences of the user based on the user selection.

In any of the examples, the method can further include: retrieving medical information associated with the medical procedure; and generating at least some of the auto-generated content based on the medical information and a medical profile of a user that performed the medical procedure.

In any of the examples, the method can further include: generating, using a machine learning model, auto-generated text for the one or more auto-generated images, wherein updating the draft medical report comprises: adding the auto-generated text associated with at least one of the one or more auto-generated images to the updated draft medical report.

According to some examples, a system includes: one or more processors programmed to perform the method of any of the examples.

According to some examples, a non-transitory computer-readable medium stores computer program instructions that, when executed, effectuate the method of any of the examples.

According to some examples, a medical device includes: one or more processors programmed to perform the method of any of the examples.

In any of the examples, the medical device can further include: an image capture device configured to capture one or more images of the medical procedure, wherein the one or more captured images comprise at least some of the one or more auto-generated images.

It will be appreciated that any of the variations, aspects, features, and options described in view of the systems apply equally to the methods and vice versa. It will also be clear that any one or more of the above variations, aspects, features, and options can be combined.

### BRIEF DESCRIPTION OF THE FIGURES

The present application will now be described, by way of example only, with reference to the accompanying drawings, in which:

FIG. 1A illustrates an example medical environment, according to some aspects.

FIG. 1B illustrates an example system for generating a medical report describing a medical procedure, according to some aspects.

FIG. 2 illustrates example timelines for capturing content depicting a medical procedure, according to some aspects.

FIG. 3 illustrates an example user interface displaying a draft medical report, according to some aspects.

FIG. 4 illustrates an example text generation process for generating text to be included in a draft medical report, according to some aspects.

FIG. 5 illustrates an example training process for training a machine learning model used for generation of a medical report, according to some aspects.

FIGS. 6A-6B illustrate examples of a draft medical report and an updated draft medical report including a user selection of suggested content, according to some aspects.

FIG. 7A illustrates example medical profiles stored in a medical profile database, according to some aspects.

FIG. 7B illustrates example machine learning models stored in model database 166, according to some aspects.

FIGS. 7C-7D illustrate an example image depicting an object associated with a medical procedure with and without annotations added, according to some aspects.

FIGS. 8-11 illustrate flowcharts describing example processes for generating a medical report describing a medical procedure, according to some aspects.

FIG. 12 illustrates an example computing system used for performing any of the techniques described herein, according to some aspects.

### DETAILED DESCRIPTION

Reference will now be made in detail to implementations and various aspects and variations of systems and methods described herein. Although several example variations of the systems and methods are described herein, other variations of the systems and methods may include aspects of the systems and methods described herein combined in any suitable manner having combinations of all or some of the aspects described.

Described are systems, methods, and programming for automating a medical report generation process. A medical report may include content describing a medical procedure. The content may include images, text, audio, and/or video relating to one or more phases of the medical procedure. As a medical procedure is performed, certain medical events may occur that are indicative of the different phases (e.g., a first incision made in a patient may be associated with one phase, whereas detection of an anatomical structure may indicate that a surgery has entered another phase). Traditionally, a user wanting to document any of these medical events in a draft medical report would manually obtain content (e.g., capture an image) describing the medical events. However, as described above, these traditional approaches may lead to medical workflow inefficiencies and can impact the quality of patient care.

One or more machine learning models may be trained to detect important medical events associated with a performance of the medical procedure and capture content depicting those medical events. One or more medical procedures referenced herein may use a medical device including one or more image capture devices, such as an endoscope, which may capture one or more videos depicting the medical procedure and present the videos to a user via one or more display devices. The trained machine learning model may analyze the one or more videos and determine whether any frames from the videos depict one or more of the medical events. The trained machine learning model may determine whether a given frame from the videos includes one or more image descriptors. An image descriptor, as described herein, can include objects, environmental factors, and/or contextual information. Different image descriptors may be associated with different medical events. For example, the presence of one or more particular objects within a frame may indicate that a particular medical event has occurred, and the medical event may be associated with a particular phase of the medical procedure. In this example, if any one or more of those objects are detected, the corresponding frame may be stored for inclusion in the draft medical report.

Medical events may be identified based on prior performances of the medical procedure. For example, prior performances of the medical procedure may be analyzed to identify images that were captured during medical events in the prior performances. Draft medical reports created to describe those prior performances of the medical procedures may include one or more images describing identified medical events. For example, these images may depict important medical events associated with a given medical procedure and therefore may indicate the type of content that should be included in a draft medical report.

One or more machine learning models may be trained to detect occurrences of medical events during subsequent performances of the medical procedure. Based on the medical events being detected, the one or more machine learning models may select one or more images depicting the medical events for a draft medical report. The machine learning models may be trained based on the prior performances of the medical procedure, mentioned above. The prior performances may include performances by one or more users (e.g., surgeons, medical professionals, etc.). Thus, the machine learning models may be configured to learn specific preferences of a given user in capturing and/or selecting images.

Training the machine learning models may include generating representations of the images captured from prior performances of the medical procedure. These image representations may translate image variables to arrays of numbers (e.g., vectors) describing semantic information about the image. The arrays may be projected into a latent space. Clusters in the latent space may indicate similarities between images. For example, each cluster may be associated with a particular medical event (e.g., a start of a certain phase).

When a video of a performance of the medical procedure is obtained, the frames of the video may be analyzed using the trained machine learning model. The trained machine learning model may generate representations of the frames and may determine whether the generated representations are similar to one or more image representations associated with the images captured during prior performances of the medical procedure. If the representations are determined to be similar, the trained machine learning model may classify that frame as depicting the same (or similar) medical event as that which is captured in the image(s) from the prior performances.

The machine learning models may further be trained to automatically generate a draft medical report. The machine learning models may determine which medical events (and thus, which images) to include in the draft medical report based on the prior performances of the medical procedure. For example, medical reports previously produced for the prior performances of the medical procedure may include an image depicting a particular medical event. The machine learning models may determine whether videos and/or images of a current performance of the medical procedure depict the same or a similar medical event. If one or more images and/or videos from the current performance of the medical procedure does depict the same (or similar) medical event, the machine learning models may extract the corresponding image and/or video from the one or more videos of the medical procedure and include the extracted image and/or video in a draft medical report. The draft medical report, including the video and/or image contents extracted from one or more videos of the current performance of the medical procedure, may be presented to a user (e.g., a surgeon who performed a medical procedure, a medical professional who assisted in the medical procedure, etc.) for review, modification, and/or finalization.

The machine learning models may further be trained to provide the user with suggested content for inclusion in the draft medical report. The suggested content can include images, text, audio, video, and/or other content that may be helpful to include in the medical report for a patient. The suggested content may be identified by the machine learning models as being relevant to the medical procedure. In particular, the suggested content may represent aspects of the medical procedure that may be different from the content identified based on the prior performances. For example, if an unexpected medical event or object is detected during the medical procedure, an image and/or video depicting the medical event or object may be provided as suggested content. For example, the suggested content may include one or more auto-generated images. The suggested content may instead or additionally include auto-generated text describing some or all of the auto-generated images. The auto-generated text may be generated using one or more of the machine learning models. The suggested content may be provided to the user with an option to select some or all of the suggested content for inclusion in the draft medical report.

It should be noted that although some aspects are described herein with respect to machine learning models, other prediction models (e.g., statistical models or other analytics models) may be used in lieu of or in addition to machine learning models (e.g., a statistical model replacing a machine-learning model and a non-statistical model replacing a non-machine-learning model).

Although one or more videos are described above as being analyzed by the trained machine learning models, persons of ordinary skill in the art will recognize that one or more images may be analyzed instead of or in addition to the one or more videos. Furthermore, a video may be split into frames prior to being provided to the trained machine learning models. As described herein, a video refers to a sequence of images (called frames). An image sensor (e.g., an image capturing device) may capture an image at a predefined cadence, and this sequence of captured images may comprise the video.

In the following description, it is to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Certain aspects of the present disclosure include process steps and instructions described herein in the form of an algorithm. It should be noted that the process steps and instructions of the present disclosure could be embodied in software, firmware, or hardware and, when embodied in software, could be downloaded to reside on and be operated from different platforms used by a variety of operating systems. Unless specifically stated otherwise as apparent from the following discussion, it is appreciated that, throughout the description, discussions utilizing terms such as “processing,” “computing,” “calculating,” “determining,” “displaying,” “generating,” or the like, refer to the action and processes of a computer system, or similar electronic computing device, that manipulates and transforms data represented as physical (electronic) quantities within the computer system memories or registers or other such information storage, transmission, or display devices.

The present disclosure in some examples also relates to a device for performing the operations herein. This device may be specially constructed for the required purposes, or it may comprise a general-purpose computer selectively activated or reconfigured by a computer program stored in the computer. Such a computer program may be stored in a non-transitory, computer readable storage medium, such as, but not limited to, any type of disk, including floppy disks, USB flash drives, external hard drives, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each coupled to a computer system bus. Furthermore, the computers referred to in the specification may include a single processor or may be architectures employing multiple processor designs for increased computing capability. Suitable processors include central processing units (CPUs), graphical processing units (GPUs), field-programmable gate arrays (FPGAs), and ASICs.

The methods, devices, and systems described herein are not inherently related to any particular computer or other apparatus. Various general-purpose systems may also be used with programs in accordance with the teachings herein, or it may prove convenient to construct a more specialized apparatus to perform the required method steps. The required structure for a variety of these systems will appear from the description below. In addition, the present application is not described with reference to any particular programming language. It will be appreciated that a variety of programming languages may be used to implement the teachings of the present application as described herein.

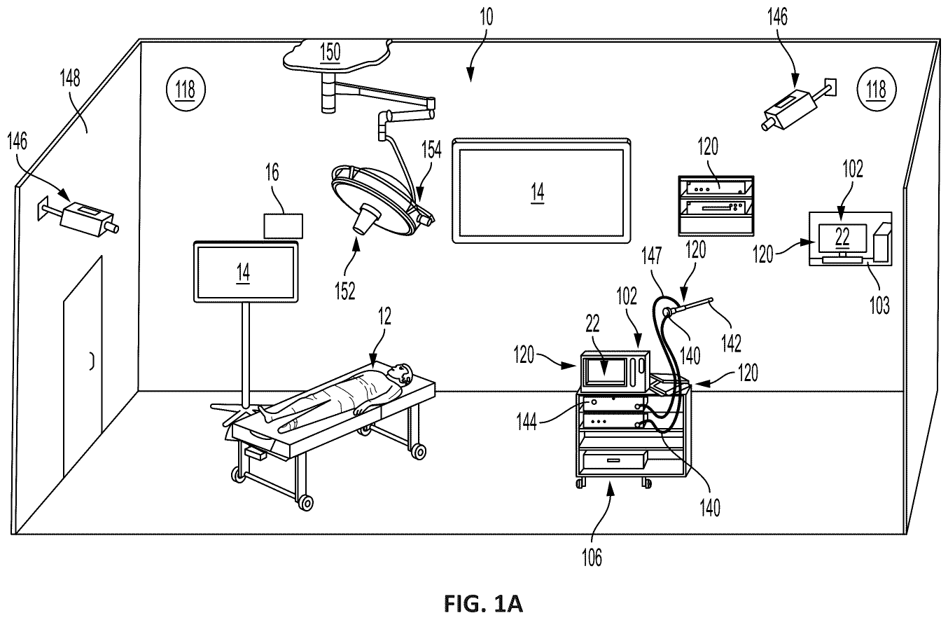

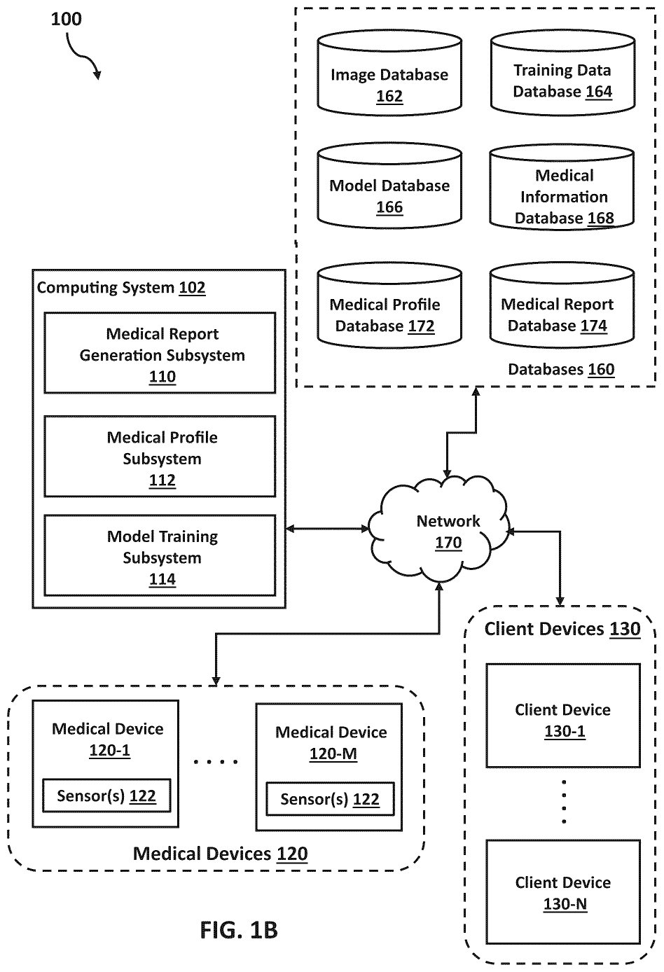

FIG. 1A illustrates an example medical environment 10, according to some aspects. Medical environment 10 may represent a surgical suite or other medical facility where a medical procedure may be performed. Medical environment 10 may include devices used to perform a medical procedure on a patient 12. Such devices may include one or more sensors, one or more display devices, one or more light sources, one or more computing devices, and/or other components. Medical environment 10 comprises at least one medical device 120 to assist in performing a medical (e.g., surgical) procedure and/or for record-keeping purposes. For example, medical device 120 may be used to input or receive patient information (e.g., to/from electronic medical records (EMRs), electronic health records (EHRs), hospital information systems (HIS), communicated in real-time from another system, etc.). The received patient information may be saved onto medical device 120. Alternatively or additionally, the patient information may be displayed using medical device 120. In some aspects, medical device 120 may be used to record patient information, including storing the information or images in an EMR, EHR, HIS, or other databases.

Medical device 120 located in medical environment 10 can include any device that is capable of saving information related to a patient 12. Medical device 120 may or may not be coupled to a network that includes records of patient 12. Medical device 120 may include a computing system 102 (e.g., a desktop computer, a laptop computer, a tablet device, etc.) having an application server. However, alternatively, one or more instances of computing system 102 may be included within medical environment. Computing system 102 can have a motherboard that includes one or more processors or other similar control devices as well as one or more memory devices. The processors may control the overall operation of computing system 102 and can include hardwired circuitry, programmable circuitry that executes software, or a combination thereof. The processors may, for example, execute software stored in a memory device. The processor may include, for example, one or more general- or special-purpose programmable microprocessors and/or microcontrollers, application specific integrated circuits (ASICs), programmable logic devices (PLDs), programmable gate arrays (PGAs), or the like. The memory device may include any combination of one or more random access memories (RAMs), read-only memories (ROMs) (which may be programmable), flash memory, and/or other similar storage devices. Patient information may be inputted into computing system 102 (e.g., for making an operative note during the medical procedure on patient 12 in medical environment 10) and/or computing system 102 can transmit the patient information to another medical device 120 (via either a wired connection or wirelessly).

Medical device 120 can be positioned in medical environment 10 on a table (stationary or portable), a portable cart 106, an equipment boom, and/or shelving 103. FIG. 1A illustrates two forms of computing system 102: a first computing system in the form of a desktop computer shelving 103 and a second computing system incorporated into portable cart 106. Further, examples of the disclosure may include any number of computer systems.

In some aspects, medical environment 10 may be an integrated suite used for minimally invasive surgery (MIS) or fully invasive procedures. Video and audio components and associated routing may be located throughout medical environment 10. The components may be located on or within the walls, ceilings, or floors of medical environment 10. Wires, cables, and hoses can be routed through suspensions, equipment booms, and/or interstitial space. The wires, cables, and/or hoses in medical environment 10 may be capable of connecting to mobile equipment, such as portable cart 106, C arms, microscopes, etc. to communicate routing audio, video, and data information.

Computing system 102 may be configured to capture images and/or video, and may route audio, video, and other data (e.g., device control data) throughout medical environment 10. Computing system 102 and/or associated router(s) may route the information between devices within or proximate to medical environment 10. In some aspects, computing system 102 and/or associated router(s) (not shown) may be located external to medical environment 10 (e.g., in a room outside of an operating room), such as in a closet. As an example, the closet may be located within a predefined distance of medical environment 10 (e.g., within 325 feet). In some aspects, computing system 102 and/or the associated router(s) may be located in a cabinet inside or adjacent to medical environment 10.

Computing system 102 may be capable of recording images and/or videos, each of which may be displayed via one or more display devices. Computing system 102, alone or in combination with one or more audio sensors, may also be capable of recording audio, outputting audio, or a combination thereof. In some aspects, patient information can be inputted into computing system 102. The patient information may be added to the images and videos recorded and/or displayed. Computing system 102 can include, or may be part of an image capture device that may include, internal storage (e.g., a hard drive, a solid state drive, etc.) for storing the captured images and videos. Computing system 102 can also display any captured or saved images (e.g., from the internal hard drive) or on an associated touchscreen monitor 22 and/or an additional monitor 14 coupled to computing system 102 via either a wired connection or wireless connection. It is contemplated that computing system 102 could obtain or create images of patient 12 during a medical procedure from a variety of sources (e.g., from video cameras, video cassette recorders, X-ray scanners (which convert X-ray films to digital files), digital X-ray acquisition apparatus, fluoroscopes, computed tomography (CT) scanners, magnetic resonance imaging (MRI) scanners, ultrasound scanners, charge-coupled (CCD) devices, and other types of scanners (handheld or otherwise)). If coupled to a network, computing system 102 can also communicate with a picture archiving and communication system (PACS), as is well known to those skilled in the art, to save images and video in the PACS and to retrieve images and videos from the PACS. Computing system 102 can couple and/or integrate with, e.g., an electronic medical records database and/or a media asset management database.

A touchscreen monitor 22 and/or an additional monitor 14 may be capable of displaying images and videos captured live by one or more image sensors within medical environment 10 (e.g., a camera head 140 coupled to an endoscope 142, which may communicate with a camera control unit 144 via a fiber optic cable 147, wires, and/or a wireless connection), and/or replayed from recorded images and videos. It is further contemplated that touchscreen monitor 22 and/or additional monitor 14 may display images and videos captured live by a room camera 146 fixed to walls 148 or a ceiling 150 of medical environment 10 (e.g., a room camera 146 as shown or a camera 152 in surgical light 154). The images and videos may be routed from the cameras to computing system 102 to touchscreen monitor 22 and/or additional monitor 14.

One or more speakers 118 may be positioned within medical environment 10 to provide sounds, such as music, audible information, and/or alerts that can be played within the medical environment during the procedure. For example, speaker(s) 118 may be installed on the ceiling and/or positioned on a bookshelf, on a station, etc.

One or more microphones 16 may sample audio signals within medical environment 10. The sampled audio signals may comprise the sounds played by speakers 118, noises from equipment within medical environment 10, and/or human speech (e.g., voice commands to control one or more medical devices or verbal information conveyed for documentation purposes). Microphone(s) 16 may be located within a speaker (e.g., a smart speaker) attached to additional monitor 14, as shown in FIG. 1A, and/or within the housing of additional monitor 14. Microphone(s) 16 may communicate via a wired or wireless connection with computing system 102. Microphones 16 may provide, record, and/or process the sampled audio signals. For example, computing system 102 may provide music to speakers 118 to be played during a medical procedure. Microphone 16 may determine the user's speech, and microphone 16 (as well as, or alternatively, a microphone of computing system 102) may record the user's speech for documentation purposes (e.g., record verbal information for educational purposes, make room calls, send real-time information to pathologists, etc.). Microphone(s) 16 may include near-field and/or far-field microphones. Microphone(s) may include a microphone array, such a MEMS microphone array. An exemplary microphone array may be distributed throughout an operating room. The microphones 16 may include a linear/circular microphone array that implements beamforming, or a set of spatially-separated distributed microphones (e.g., installed at various locations in the operating room). Additionally or alternatively, computing system 102 may be capable of modifying the audio signals, including recognizing the voice commands received from the user (e.g., surgeon, medical professional, etc.). In some aspects, computing system 102 may correspond to an operating room (OR) hub. The OR hub may be configured to process the audio signals, including recognizing the voice commands.

FIG. 1B is a diagram illustrating an example system 100, according to some aspects. System 100 may include computing system 102, medical devices 120 (e.g., medical devices 120-1, 120-M), client devices 130 (e.g., client device 130-1, 130-N), databases 160 (e.g., image database 162, training data database 164, model database 166, medical information database 168, medical profile database 172, medical report database 174), or other components. Components of system 100 may communicate with one another using network 170 (e.g., the Internet).

Medical devices 120 may include one or more sensors 122, such as an image sensor, an audio sensor, a motion sensor, or other types of sensors. Sensors 122 may be configured to capture one or more images, one or more videos, audio, or other data relating to a medical procedure. Medical device 120 may use sensors 122 to obtain or create images of patient 12 during a medical procedure from a variety of sources (e.g., from video cameras, video cassette recorders, X-ray scanners (which can convert X-ray films to digital files), digital X-ray acquisition apparatus, fluoroscopes, computed tomography (CT) scanners, magnetic resonance imaging (MRI) scanners, ultrasound scanners, charge-coupled (CCD) devices, and other types of scanners (handheld or otherwise)). For example, medical device 120 may capture images and/or videos of an anatomical structure of patient 12. As another example, medical device 120 may be a medical imaging device (e.g., MRI machines, CT machines, X-Ray machines, etc.). As yet another example, medical device 120 may be a biometric data capture device (e.g., a blood pressure device, pulse-ox device, etc.).

Client devices 130-1 to 130-N may be capable of communicating with one or more components of system 100 via a wired and/or wireless connection (e.g., network 170). Client devices 130 may interface with various components of system 100 to cause one or more actions to be performed. For example, client devices 130 may represent one or more devices used to display images and videos to a user (e.g., a surgeon, medical professional, etc.). Examples of client devices 130 may include, but are not limited to, desktop computers, servers, mobile computers, smart devices, wearable devices, cloud computing platforms, display devices, mobile terminals, fixed terminals, or other client devices. Each client device 130-1 to 130-N of client devices 130 may include one or more processors, memory, communications components, display components, audio capture/output devices, captured image components, other components, and/or combinations thereof.

Computing system 102 may include one or more subsystems, such as medical report generation subsystem 110, medical profile subsystem 112, model training subsystem 114, or other subsystems. Subsystems 110-114 may be implemented using one or more processors, memory, and interfaces. Distributed computing architectures and/or cloud-based computing architectures may alternatively or additionally be used to implement some or all of the functionalities associated with medical report generation subsystem 110, medical profile subsystem 112, and/or model training subsystem 114.

It should be noted that while one or more operations are described herein as being performed by particular components of computing system 102, those operations may be performed by other components of computing system 102 or other components of system 100. As an example, while one or more operations are described herein as being performed by components of computing system 102, those operations may alternatively be performed by one or more of medical devices 120 and/or client devices 130.

Medical report generation subsystem 110 may be configured to generate a draft medical report describing a medical procedure. As used herein, the term “draft medical report” is used to refer to a medical report that has not been finalized yet. The finalization may occur based on a user selection to finalize the report. Prior to finalization, the draft medical report may be updated to incorporate new content, remove content, modify content, or perform other operations. After being finalized, the medical report may be stored for later review with a patient, medical professionals, and/or other individuals permitted to view the medical report. The medical report may be updated with data obtained at later points in time. For example, medical images captured during follow-up appointments may be added to the medical report to track a patient's progress.

A user associated with a medical procedure (e.g., a surgeon, medical professional, etc.) may be identified by computing system 102 and a medical profile for the user may be retrieved from medical profile database 172. For example, medical report generation subsystem 110 may be configured to determine the identity of the user. Medical report generation subsystem 110 may determine the user based on credentials input by the user (e.g., via client device 130). For example, an identity of the user may be determined based on credentials of the user (e.g., an RFID tag, a device detected that is associated with the user, retinal scan, facial scan, fingerprinting, manual input, or other techniques). The user may alternatively or additionally be identified based on scheduling data associated with the user and/or the medical procedure. Based on the input credentials, medical report generation subsystem 110 may provide a user identifier to medical profile subsystem 112.

Medical profile subsystem 112 may be configured to identify, generate, update, and/or retrieve a medical profile of a user performing a medical procedure (e.g., a surgeon, medical staff, etc.). The medical profile may refer to a surgical profile that comprises preferences of the user (e.g., a surgeon, medical staff, etc.). These preferences may indicate medical events that occur during a medical procedure (e.g., image characteristics indicative of the medical events) and may be documented via one or more images, videos, audio, or other data. The preferences may instead or additionally indicate preferences related to the type of content to be included in a draft medical report for the medical procedure, the times at which content is captured during the medical procedure, the manner in which content is to be presented in the draft medical report, additional content that may be suggested for inclusion in the draft medical report, or other information.

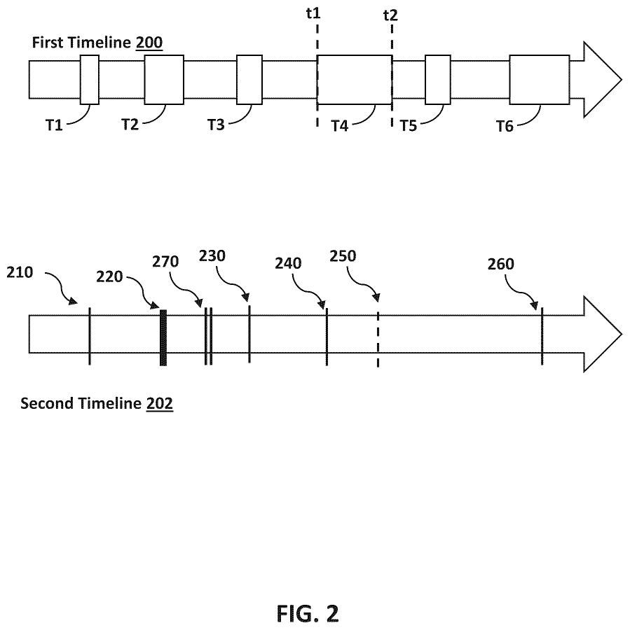

For example, the medical profile of a user may store indications of time windows, such as time windows T1-T6 illustrated in FIG. 2, that medical events are expected to occur during the medical procedure.

FIG. 2 illustrates example timelines for capturing, or otherwise generating, content (referred to herein as “auto-generated content”) depicting medical events during a medical procedure, according to some aspects. In some aspects, auto-generated content may include auto-generated images (e.g., images captured automatically during time windows corresponding to ranges of time within which medical events are expected to occur) and/or auto-generated text (further described below). As shown in FIG. 2, first timeline 200 may include time windows T1-T6, each of which may correspond to ranges of times within which certain medical events associated with the medical procedure are expected to occur. These medical events may relate to particular phases of the medical procedure that are to be documented and included within a draft medical report describing the medical procedure (e.g., a preoperative phase, an intraoperative phase, a postoperative phase, etc.). As mentioned above, a medical event may comprise one or more objects or actions being detected within images of the medical procedure. For example, a particular anatomical structure may be detected within a video feed of a minimally invasive surgical procedure captured using an endoscope, and detection of the anatomical structure may indicate that a particular phase of the medical procedure has started (or ended). Time windows T1-T6 may be determined using information comprising ranges of times during which the medical events typically occurred during prior performances of the medical procedure. For example, an image depicting a particular anatomical structure may have been previously captured during prior surgeries at various times between a first time point (e.g., t1) and a second time point (e.g., t2). Therefore, it can be expected that the particular anatomical structure may be detected during subsequent performances of the medical procedure at least during a time window starting at first time point t1 and ending at second time point t2, where first time point t1 may represent a start of one of time windows T1-T6 (e.g., T4, as illustrated in FIG. 2) and second time point t2 may represent an end of the same time window (e.g., T4).

A second timeline 202 may indicate when content was captured during the medical procedure (e.g., automatically and/or manually such as by camera head 140 of endoscope 142 illustrated in FIG. 1A). Times 210-270 may indicate when an image, video, audio, text, and/or other content was captured. For example, at times 210, 230, 240, and 260, medical events expected to occur during time windows T1, T3, T4, and T6, respectively, may be detected, and content depicting those medical events may be captured. In another example, at time 220, content may be captured depicting medical events such as an unexpected object, action, and/or other event that occurred during the medical procedure. In another example, at time 250, content may be captured based on a manual user selection (e.g., based on a user invoking an image capture option). In another example, at time 270, devices (e.g., microphones 16, touchscreen 22, room camera 146, etc., shown in FIG. 1A) disposed within medical environment 10 may detect an input, such as audio signals, text, gestures, or other content. For example, a user may utter a comment, and audio signals of the utterance may be captured via one or more audio sensors (e.g., microphones 16). Audio data representing the utterance may be associated with other content captured during the medical procedure. For example, audio data representing the utterance may be associated with content (e.g., an image) captured at time 220 and/or at time 240, at least because the contents were captured during the same time window (e.g., T2).

Optionally, timelines 200 and 202 may only run while an image sensor (e.g., camera head 140 of endoscope 142 of FIG. 1A) is capturing images of a surgical site, such as inside a patient's body. For example, the detection (e.g., by medical report generation subsystem 110) of the endoscope 142 being removed from the patient's body (e.g., for lens cleaning) may pause timelines 200 and 202, suspending the auto-generation of content and the recording of time. The detection (e.g., by medical report generation subsystem 110) of the image sensor being reinserted into the patient's body may resume timelines 200 and 202, resuming the auto-generation of content and the recording of time. In another example, timelines 200 and 202 may be paused based on a manual user indication that the image sensor has been removed from the patient's body and may be resumed based on a manual user indication that the image sensor has been returned to the patient's body.

Pausing timelines 200 and 202 when the image sensor is outside of the patient's body may improve the accuracy of a generated draft medical report. For example, pausing timelines 200 and 202 while the image sensor has been removed from the patient's body during a time window within which certain medical events are expected to occur (e.g., T1) may prevent the time window from ending prematurely (e.g., before the occurrence of medical events that are to be documented within that window) due to the elapsed time in which the image sensor is removed. If the time window ends prematurely, the medical events that are to be documented within that window may not be captured or may be associated with an incorrect time window or phase of the medical procedure. Preventing the elapsed time during which the image sensor is removed from counting against the time window may prevent the time window from ending prematurely and may allow the auto-capture of all the medical events that are to be documented within that time window, improving accuracy of the generated draft medical report. Improving accuracy of the draft medical report may reduce the time a user has to spend in verifying the generated draft medical report.

Additionally or alternatively, pausing timelines 200 and 202 when the image sensor is outside of the patient's body, thereby suspending the auto generation of content, may prevent capturing of unwanted data in a draft medical report. For example, suspending the auto-capture of images while the image sensor is outside of the patient's body may prevent the capture of images containing protected health information (PHI) or personally identifiable information (PII). Additionally or alternatively, suspending the auto-capture of images while the image sensor is outside of the patient's body may prevent the capture of images irrelevant to the draft medical report. Preventing the capture of unwanted data may reduce the amount of time a user has to spend verifying the generated draft medical report.

Returning to FIG. 1A, medical profile subsystem 112 may be configured to generate some or all of the auto-generated content for a draft medical report based on the medical profile of the user and/or medical information associated with the medical procedure. The medical information may be retrieved from medical information database 168. The medical information may be associated with a medical workflow. For example, the medical information may indicate stages included in one or more medical workflows, content (or pointers to content) associated with the stages of the medical workflows, or other information. The medical information may be associated with patient 12 (shown in FIG. 1A). For example, the medical information may include medical images of patient 12 captured prior to the medical procedure being performed, medical exam results of patient 12 determined prior to the medical procedure, or other information. The medical information may instead or additionally include subsequent medical images and medical exam results obtained for patient 12 subsequent to the medical procedure being performed. For example, medical images of patient 12 captured after the medical procedure may be used to update a medical report describing the medical procedure and/or patient 12.

Model training subsystem 114 may be configured to train one or more machine learning models, such as those stored in model database 166. Some or all of these machine learning models may be used to generate a draft medical report. For example, a machine learning model may be trained to detect certain medical events that transpire during the medical procedure and cause one or more images, video, audio, and/or other content of the medical events to be captured. The content may be automatically identified, thereby relieving a user associated with the medical procedure from having to manually capture and/or select the images and/or videos for the draft medical report.

The machine learning models may be trained to analyze videos of the medical procedure obtained from a medical device 120, such as an endoscope, and determine whether frames from the videos include image descriptors associated with the medical events. For example, prior performances of the medical procedure may be analyzed to identify image descriptors typically detected within videos of the medical procedure. Frames of the video determined to include one or more of the image descriptors may be analyzed using the machine learning models to determine whether the detected image descriptors are the same or similar to image descriptors identified from the prior performances of the medical procedure. If the detected image descriptors are the same or similar, it may be determined that one or more medical events has been detected. Based on this determination, the machine learning models may notify medical report generation subsystem 110 and/or cause medical report generation subsystem 110 to select the corresponding frames depicting the medical event for a draft medical report. The medical report generation subsystem 110 may include some or all of these selected frames within a draft medical report.

Image descriptors may include objects, environmental factors, and/or contextual information associated with particular phases of the medical procedure. For example, the objects that may be depicted within frames of the video may include patient 12, a user associated with the medical procedure (e.g., a surgeon, medical staff, etc.), medical devices 120, monitor 14, touchscreen 22, medical equipment (e.g., surgical sponges, scalpels, retractors, etc.), fluids (e.g., water, saline, blood, etc.), or other objects. In another example, the environmental factors may include a temperature, humidity level, noise level, ambient light level, and/or other environmental factors related to medical environment 10 (shown in FIG. 1A). In another example, the contextual information may include a sponge count, a volume of blood loss, cardiovascular/respiratory information of patient 12, or other contextual information.

The machine learning models may analyze videos of the medical procedure obtained from a camera (e.g., room camera 146) and/or tracking information obtained by a tracking system (e.g., an image-based tracking system and/or a motion sensor-based tracking system communicatively coupled to computing system 102) to track an object (e.g., medical device 120) over time. For example, the machine learning models could detect one or more of the object's use, location, or path over time using computer vision analysis. Auto-generated content (e.g., videos, images, text, etc.) describing one or more of a tracked object's use, location, or path over time may be provided to the user with the draft medical report as suggested content.

The detection of medical events may instead or additionally be based on the detection of other image descriptors (e.g., unexpected objects, actions, details, etc.) typically captured by users who have performed the medical procedure. For example, a medical event detected by the trained machine learning models may comprise an anatomical structure with an unexpected abnormality, a medical event detected during a different time window than expected (e.g., described above with reference to FIG. 2), etc. As described in greater detail below, auto-generated images depicting the unexpected event may be provided to the user with the draft medical report as suggested content.

Additionally or alternatively, the detection of medical events may be based on a detection of a change in the operation of a device (e.g., medical device 120). For example, if a medical device is a device with a software control that may be adjusted (e.g., a burr for shaving bone with an adjustable speed control), a medical event detected by the trained machine learning models may include a change in the software control (e.g., increasing or decreasing the speed of the burr). In another example, a medical event detected by the trained machine learning models may include a warning message created by a medical device (e.g., medical device 120). Auto-generated content describing the medical event and/or the change in operation of a device may be provided to the user with the draft medical report as suggested content.

The machine learning models may cause and/or instruct an image sensor (e.g., camera head 140 of endoscope 142 of FIG. 1A) to capture images depicting medical events in a medical procedure that have previously been determined as important for a medical report. This determination may be made based on prior performances of the medical procedure. In particular, the determination may be made based on prior performances of the medical procedure by a particular user (e.g., stored in a medical profile of the user, as described above). Therefore, the machine learning models may be trained to detect and document medical events in a medical procedure based on user preferences related to the medical procedure. The medical profile of the user (e.g., stored in medical profile database 172) may include entries referencing one or more machine learning models that were trained using images from prior medical procedures performed by the user. Thus, different machine learning models may be trained for different users, as well as for different medical procedures. For example, a given user's medical profile may store and/or indicate where to access (e.g., via a pointer) a first machine learning model trained to detect medical events during a performance of a first medical procedure by the user, a second machine learning model trained to detect key events during a performance of a second medical procedure by the user, etc.

The machine learning models may additionally or alternatively be trained to generate text, graphics, or other descriptive information (e.g., hereinafter collectively referred to as “auto-generated text”) corresponding to the captured content. In addition, the machine learning models may be trained to associate user-provided text with auto-generated text, merge the auto-generated text with the user-provided text, and generate a draft medical report based on the merged text, the auto-generated content, and/or other information.

The machine learning models described herein may be trained to automatically create a draft medical report. The draft medical report may include auto-generated content, such as images, videos, audio, graphics, text, etc. For example, the auto-generated content may include images automatically captured during the medical procedure that depict some or all of the aforementioned medical events. The draft medical report may include one or more of images, videos, or text indicating the detected use, location, and/or path of a tracked object (e.g., device 120) over time. In addition, the draft medical report may include any images and/or video captured during the medical procedure based on a user instruction (e.g., manually captured). For example, the user may identify a medical event in the medical procedure that should be included in the draft medical report and may manually capture an image and/or video of the medical event. These manually captured images, videos, or other content may be automatically included in the draft medical report along with the auto-generated content. The auto-generated content in the draft medical report may include suggested content. For example, the suggested content may include one or more auto-generated images and/or auto-generated text describing the one or more auto-generated images. In one example, the draft medical report may include at least one suggested image and suggested text describing the at least one suggested image.

The machine learning models that may be used to automatically create a draft medical report can include one or more large language models (LLM). The one or more LLMs may be trained on various information sources, including but not limited to intraoperative images, medical literature, procedural knowledge, and/or medical guidelines. The one or more LLMs may be trained to automatically create a draft medical report or portion thereof based on various information, including but not limited to, one or more audio feeds, patient monitoring data (e.g., vital signs), surgical phase information, details of a surgical procedure, and/or environmental factors. For example, an audio feed, patient monitoring data, surgical phase information, details of a surgical procedure, and/or environmental factors can be converted into text, which is then provided to the LLM. The LLM may process the converted text to identify key points in the surgical procedure and summarize the information into a suitable draft medical report. The LLM may be trained to attenuate noise and/or any other unwanted ambient sounds converted in the text (e.g., sounds of medical devices, peripheral utterances of medical staff, and the like).

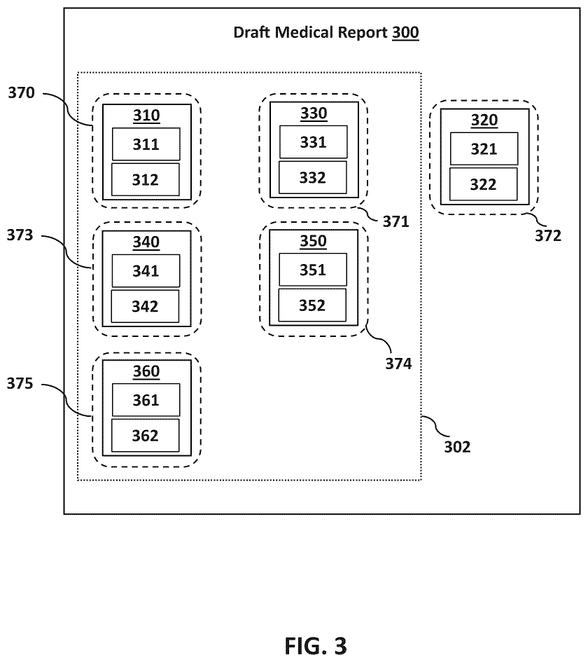

FIG. 3 illustrates an example draft medical report 300, according to some aspects. Draft medical report 300 may be presented to a user (e.g., surgeon, medical staff, etc.) via client device 130. For example, a user interface including draft medical report 300 may be rendered on a display screen of client device 130. Draft medical report 300 may include auto-generated content, such as auto-generated content 310, 330, 340, 350, and 360 and suggested content 320. Auto-generated content 310, 330, 340, 350, and 360, and suggested content 320 may include images, video, text, audio, graphs, or other types of content. Area 302 may indicate which content will be included in a finalized version of draft medical report 300. For example, if draft medical report 300 as illustrated in FIG. 3 is finalized, suggested content 320 may not be included in the medical report whereas auto-generated content 310, 330, 340, 350, and 360 may be included.

The draft medical report 300 can be used in a number of ways, including, for example, for post-operative analysis in which a user (e.g., surgeon or other medical personnel) may identify potential areas for improvement and may use various analytics and/or recommendations included in the draft medical report 300 to reduce the risk of medical errors. The draft medical report 300 may be used for training, such as to train medical staff, students, etc. on a real-life medical procedure while maintaining patient privacy. The draft medical report 300 may be used for research. For example, data from multiple surgeries may be aggregated and analyzed to identify patterns, trends, and/or best practices, which may lead to advancements in surgical techniques and/or improved patient outcomes. The draft medical report 300 may be used for determining root causes of adverse events or unexpected complications, such as to support an investigation during the course of a medical malpractice lawsuit.

Suggested content 320 may represent content selected by medical report generation subsystem 110 based on one or more medical report criteria. Likewise, medical report generation subsystem 110 may be configured to assign the auto-generated content to different regions of draft medical report 300 based on the medical report criteria. The medical report criteria may be determined based on a type of medical procedure being performed. For example, a first type of medical procedure may be associated with first medical report criteria, while a second type of medical procedure may be associated with second medical report criteria. The medical report criteria may instead or additionally be determined based on a medical profile of a user (e.g., stored in medical profile database 172). For example, the medical report criteria may include preferences of the user provided in the medical profile of the user. The user's preferences for creating a medical report may include indications of the types of content the user prefers to include in the medical report, a style of font and/or layout of the medical report, or other preferences. The medical profile of the user may include a template comprising one or more of the aforementioned user preferences.

Medical report generation subsystem 110 may select content (e.g., one or more images) captured during the medical procedure, and may assign that content to particular regions of draft medical report 300. The types of content selected and/or the location that content is assigned within draft medical report 300 may be based on the medical report criteria (e.g., a user ID, procedure preferences, model preferences, report preferences, etc.). As an example, draft medical report 300 may include regions 370, 371, 373, 374, and 375. Auto-generated content may be selected from content captured during the medical procedure and automatically assigned to one of regions 370-375. For example, auto-generated content 310, 330, 340, 350, and 360 may be assigned to regions 370, 371, 373, 374, and 375, respectively.

As mentioned above, the auto-generated content may include suggested content, such as suggested content 320. Medical report generation subsystem 110 may identify suggested content 320 (e.g., an image) and may assign suggested content 320 to region 372, where it may be presented as a suggestion for inclusion within the medical report. Auto-generated content 310, 330, 340, 350, and 360 may correspond to content the above-described machine learning models of system 100 determined should be included within draft medical report 300 (e.g., at least because the content depicts particular medical events to be documented). On the other hand, suggested content 320 may correspond to content that the machine learning models of system 100 determined may be useful to include within draft medical report 300 (e.g., content which depicts an unexpected medical event that may be important to document).

The suggested content may instead or additionally include content obtained from previously performed medical procedures. This content may correspond to content captured during prior performances of the medical procedure by the user viewing draft medical report 300 (e.g., the surgeon, medical professional, etc.) and/or during prior performances of the medical procedure by other users (e.g., other surgeons, medical professionals, etc.). For example, draft medical report 300 may optionally include suggested content, such as one or more auto-generated images (with or without corresponding auto-generated text) from prior medical procedures. The auto-generated content may be selected based on the user performing the medical procedure, the type of medical procedure, and/or a medical information of a patient to whom the medical procedure is performed. For example, the auto-generated images may include images of medical procedures of the same type performed on patients with a similar medical profile as the patient to whom the medical procedure is being performed. Patients can be grouped using any of one or more clustering algorithms (e.g., K-means clustering algorithm, density-based spatial clustering algorithms, etc.) based on their age, gender, co-morbidities, medical intervention history, etc. Medical report generation subsystem 110 may automatically retrieve (e.g., from image database 162) corresponding images and/or text from past reports of patients from the same cluster who have previously undergone the same medical procedure. Data representing the images and/or text may include information about the patients' outcomes, post-operative treatments, or other information. The user (e.g., surgeon, medical professional, etc.) can subsequently decide whether to include one or more of the suggested images in draft medical report 300. For example, images of other patients who underwent the same medical procedure may be included in draft medical report 300 for comparison purposes, such as to better support a user's decisions, findings, and/or treatment recommendations for the current patient.

Medical report generation subsystem 110 may select content to present in some or all of regions 370-375 (e.g., images captured during the medical procedure). For example, auto-generated content 310, 330, 340, and 360—which may respectively correspond to content automatically captured at times 210, 230, 240, and 260, (shown in FIG. 2)—may be automatically selected for inclusion in draft medical report 300. These images may have been selected based on one or more machine learning models detecting certain medical events. Additionally, auto-generated content 350 may be included in draft medical report 300. Auto-generated content 350 may correspond to content captured at time 250. In an example, auto-generated content 350 may comprise content that was manually captured. For instance, a user may invoke a content capture option, as described above with respect to FIG. 2 (e.g., an image capture option) to capture auto-generated content 350.

Although draft medical report 300 includes six regions (e.g., regions 370-375), persons of ordinary skill in the art will recognize that this is an example, and other example user interfaces may include more or fewer regions, or other arrangements, to present content. Furthermore, the shape, size, formatting, arrangement, or other presentation aspects of draft medical report 300 may differ and the illustrated example should not be construed as limiting.

Each of auto-generated content 310, 330, 340, 350, 360 and suggested content 320 may include one or more images and text describing the one or more images. For example, auto-generated content 310 may include an image 311 and auto-generated text 312; suggested content 320 may include auto-generated image 321 and auto-generated text 322; auto-generated content 330 may include an image 331 and auto-generated text 332; auto-generated content 340 may include an image 341 and auto-generated text 342; auto-generated content 350 may include an image 351 and auto-generated text 352; and auto-generated content 360 may include an image 361 and auto-generated text 362. Auto-generated text 312 332, 342, 352, and 362 may describe features of images 311, 331, 341, 351, and 361, respectively. Auto-generated text 322 may describe features of auto-generated image 321. As described in greater detail below, one or more machine learning models may be trained to generate text based on an image (e.g., images 311, 331, 341, 351, and 361, and auto-generated image 321) using image descriptors detected within the image.

Draft medical report 300 may also include patient metadata, such as medical information of the patient. The medical information may be used to generate auto-generated text 312, 322, 332, 342, 352, and 362 may describe features of images 311, 321, 331, 341, 351, and 361, respectively. As an example, if the medical procedure relates to the removal of an anatomical structure (e.g., a tumor), the patient's medical information may include an indication of a size of the anatomical structure. In the case of tumor removal, additional detail could be included for tracking the tumor margins removed, which could be used for determining if changes should be made in future procedures based on patient outcomes. Additionally or alternatively, draft medical report 300 may also include object tracking information, such as information regarding the detected use, location, and/or path of a tracked object (e.g., medical device 120) over time. The object tracking information may be used to generate auto-generated text 312, 322, 332, 342, 352, or 362, which may describe features of images 311, 321, 331, 341, 351, or 361, respectively.

Medical report generation subsystem 110 may be configured to generate data associated with auto-generated content 310, 320, 330, 340, 350, and 360. For example, as mentioned above, medical report generation subsystem 110 may generate text (e.g., auto-generated text 312, 322, 332, 342, 352, 362). Medical report generation subsystem 110 may instead or additionally generate graphical indicators, video links, medical information links, audio, or other content not illustrated in FIG. 3.

As mentioned above, the auto-generated content may include suggested content (e.g., suggested content 320), which one or more machine learning models determined should be suggested to the user for inclusion in draft medical report 300. For example, the machine learning models may detect an unexpected event, object, action, change in the operation of a medical device (e.g., medical device 120), warning generated by a medical device, or other potentially important aspect of the medical procedure, and may determine that documentation of this aspect should be suggested in draft medical report 300. For example, at time 220 in FIG. 2, an unexpected anatomical structure may be detected. Medical report generation subsystem 110 may be configured to select and present suggested content for inclusion in draft medical report 300, including auto-generated text, user-provided text, audio, video, or other forms of content.

The auto-generated content 310, 320, 330, 340, 350, and 360 may be included in draft medical report 300 based on factors associated with each corresponding image (e.g., image 311 of auto-generated content 310, auto-generated image 321 of suggested content 320, etc.). The factors may include objects detected within an image and/or an indication of a user input to manually capture an image. For example, one or more of regions 370-375 may present an image captured in response to a user input (e.g., a user pressed an image capture button, a user uttered a voice command to capture an image, a user performed a gesture to cause an image to be captured, etc.).

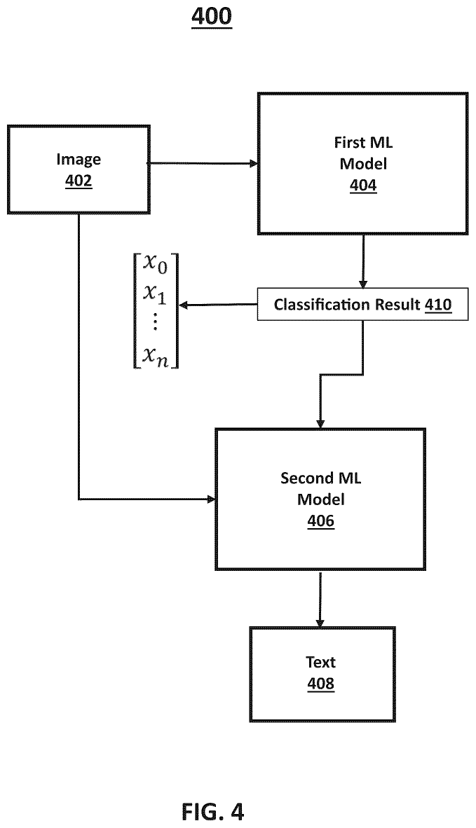

As mentioned above, auto-generated text 312, 322, 332, 342, 352, and 362 may be generated using one or more machine learning models. For example, FIG. 4 illustrates a process 400 for generating text 408 describing an image 402. In some examples, image 402 may depict a phase of a medical procedure. It is to be understood that image 402 in process 400 of FIG. 4 may represent one of images 311, 321, 331, 341, 351, or 361 in FIG. 3 and/or additional or alternative images not illustrated herein. Furthermore, although a single image, image 402, is depicted in FIG. 4, multiple images may be used to generate text 408. Image 402 may be obtained from medical device 120, as shown in FIG. 1B. Medical device 120 may include one or more sensors 122, such as an image sensor, configured to capture images and/or video of a medical procedure. For example, some of the captured images/video may depict anatomical structures of a patient (e.g., patient 12 shown in FIG. 1A). The captured images/video may be displayed to a user, such as a surgeon performing the medical procedure, via a display device (e.g., touchscreen monitor 22 and/or an additional monitor 14). For example, a real-time video feed may be displayed on touchscreen monitor 22 and/or additional monitor 14. The real-time video feed may depict intra-operative video captured by an endoscope during a minimally invasive medical procedure.

In process 400, image 402 may be provided to a first machine learning model 404. First machine learning model 404 may be configured to analyze images (e.g., frames from a real-time video feed, individual frames captured, etc.) and determine whether image descriptors associated with phases of the medical procedure (e.g., preoperative phase, intraoperative phase, postoperative phase, etc.) and/or objects of interest are depicted therein. As described above, the image descriptors may include objects of interest (e.g., anatomical structures of a patient, humans performing surgical activities, etc.), environmental factors (e.g., a temperature of medical environment 10), and/or contextual information associated with the phases of the medical procedure (e.g., a sponge count, blood loss volume, cardiovascular information, etc.). First machine learning model 404 may be a computer vision model trained to detect image descriptors, from an image and classify the image into one or more categories based on the image descriptors. For example, each category may be associated with a particular phase of the medical procedure. First machine learning model 404 may analyze image 402 and facilitate the selection of one or more of image 402 based on a determination that the image depicts one or more of the image descriptors. For example, first machine learning model 404 may analyze frames from a surgical video feed (e.g., image 402) in real-time to determine whether any anatomical structures associated with one or more phases of a medical procedure are present within the surgical video feed. The content from the video surgical feed may continually be analyzed, stored, and/or purged from memory. If an anatomical structure associated with a given phase of the medical procedure is detected in image 402, first machine learning model 404 image 402 may facilitate select the image for the draft medical report.

As mentioned above, model training subsystem 114 may be configured to train machine learning models (e.g., first machine learning model 404) using training data stored in training data database 164. The training data may include a set of images and/or videos captured during a medical procedure. The training data may further include labels indicating whether the set of images and/or videos depict one or more image descriptors from a predefined set of image descriptors classified as being associated with a medical procedure. For example, as mentioned above, the image descriptors may include objects, environmental factors, or contextual information associated with phases of the medical procedure. A phase of a medical procedure may be identified based on the image descriptors depicted by an image of the medical procedure. For example, detection of a particular anatomical structure within a frame from a surgical video feed may indicate that the medical procedure has entered a particular phase. Metadata indicating the phase may be associated with the frame, thereby enabling the frame to be identified for possible inclusion in the draft medical report.

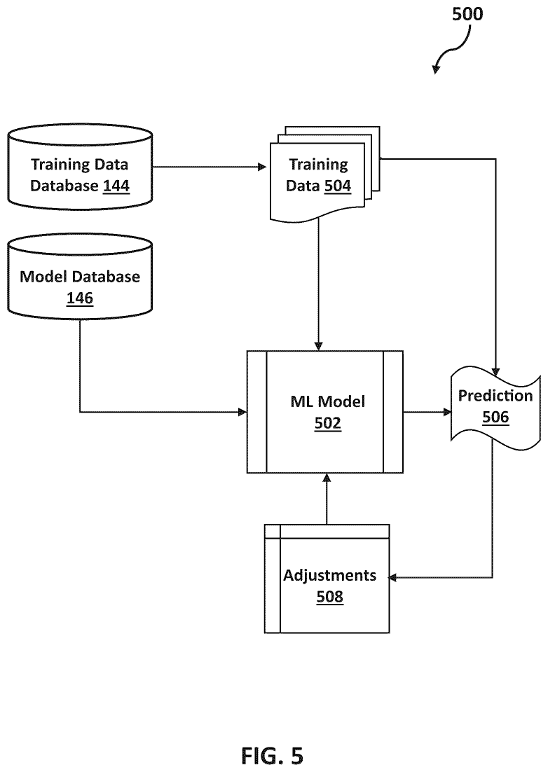

As an example, with reference to FIG. 5, model training subsystem 114 may be configured to perform a training process 500 to train a machine learning model 502. Machine learning model 502 may refer to any one or more machine learning models described herein (e.g., first machine learning model 404, second machine learning model 406, etc.) and used to generate a draft medical report. Model training subsystem 114 may select a to-be-trained machine learning model 502, which may be retrieved from model database 166. Machine learning model 502 may be selected based on criteria such as the medical procedure with which it is to be used, the user associated with the medical procedure, and/or other criteria. Model training subsystem 114 may select training data 504, which may be retrieved from training data database 164. Model training subsystem 114 may select training data 504 from training data stored in training data database 164 based on the type of machine learning model that was selected.

Model training subsystem 114 may provide training data 504 to machine learning model 502. Training data 504 may include images depicting one or more anatomical structures associated with one or more phases of a medical procedure. For example, training data 504 may include a first image depicting a first anatomical structure indicative of a beginning of a first phase of a medical procedure, a second image depicting a second anatomical structure indicative of an end of a second phase of a medical procedure, etc. Training data 504 may be provided as input to machine learning model 502, which may generate a prediction 506. Prediction 506 may indicate, amongst other information, (i) whether machine learning model 502 identified any of anatomical structures in the images and (ii) if so, a classification result indicating which anatomical structures machine learning model 502 identified. These anatomical structures may be specific to the medical procedure. For example, during a first medical procedure, a first anatomical structure being detected may be indicative of a beginning of a first phase of the first medical procedure. However, during a second medical procedure, detection of the first anatomical structure may not be indicative of a beginning of a first phase of the second medical procedure.

Prediction 506 may be compared to a ground truth identified from training data 504. As mentioned above, the images included in training data 504 may include labels. These labels may indicate anatomical structures depicted by a respective image. Model training subsystem 114 may be configured to compare labels in a given image identified as a ground truth with prediction 506 for the corresponding image. Based on the comparison, model training subsystem 114 may determine one or more adjustments 508 to be made to one or more hyperparameters of machine learning model 502. The adjustments to the hyperparameters may improve predictive capabilities of machine learning model 502. For example, based on the comparison, model training subsystem 114 may adjust weights and/or biases of one or more nodes of machine learning model 502. Process 500 may repeat until an accuracy of machine learning model 502 reaches a predefined accuracy level (e.g., 95% accuracy or greater, 99% accuracy or greater, etc.), at which point machine learning model 502 may be stored in model database 166 as a trained machine learning model. The accuracy of machine learning model 502 may be determined based on a number of correct predictions (e.g., prediction 506).

Returning to FIG. 4, first machine learning model 404 may generate a classification result 410 indicating which, if any, image descriptors (e.g., objects, environmental factors, contextual information) were detected within image 402. For example, classification result may be an n-dimensional array including classification scores x₀-xₙ. Classification scores x₀-xₙ may indicate a likelihood that a given image of image 402 depicted one (or more) of n image descriptors associated with a particular medical procedure. In one example, classification scores x₀-xₙ may each be a number between 0 and 1, where a score of 0 may indicate that first machine learning model 404 did not identify a given image descriptors of the n image descriptors within image 402, and where a score of 1 may indicate that first machine learning model 404 identified, with 100% confidence, a given image descriptor of the one or more of the n image descriptors within image 402. First machine learning model 404 may store classification result 410 with image 402 in image database 162. For example, image data representing image 402 may be updated to include metadata indicating one or more image descriptors depicted within image 402. First machine learning model 404 may output classification result 410 and the classification result 410 may be provided to a second machine learning model 406.

Second machine learning model 406 may be configured to generate text 408 describing image 402. Text 408 may be generated based on image 402, classification result 410, and/or a combination of both. In an example, an image-classification result pair comprising the classification result 410 obtained from first machine learning model 404 and the corresponding image 402 may be provided as input to second machine learning model 406. Text 408 may describe image 402, including details regarding image descriptors detected within image 402. For example, text 408 may describe medical objects identified within image 402, characteristics of the medical objects (e.g., size, weight, coloring, etc.), an identified phase of the medical procedure associated with the identified medical objects, etc. Second machine learning model 406 may include natural language processing functionalities, which may be used to generate text 408.

Second machine learning model 406 may employ pre-generated text associated with different image descriptors. For example, when a particular anatomical structure is detected, pre-generated text describing the anatomical structure may be selected from a medical lexicon. Text 408 may include some or all of the pre-generated text. Second machine learning model 406 may alternatively or additionally be configured to generate prose describing the image descriptors (if any) detected in image 402. For example, second machine learning model 406 may be a generative model that generates prose based on features included within an input image. Second machine learning model 406 may be trained to generate text using a similar process as process 500 described above. In addition to being trained with training data including images (as described above with respect to training first machine learning model 404), the training data for second machine learning model 406 may include pre-generated text for the images. In this example, prediction 506 (shown in FIG. 5) may include prose generated for a given image, which may then be compared to the pre-generated text in the training data for the given image. Model training subsystem 114 may determine adjustments 508 based on a comparison of the generated prose in prediction 506 with the pre-generated text for the image descriptors detected for the given image.

Second machine learning model 406 may be configured to receive one or more additional inputs for generating text 408 not explicitly illustrated in FIG. 4. For example, details relating to the medical procedure and/or specifics regarding the image descriptors detected (e.g., medical objects), may be provided as input to second machine learning model 406 to generate text 408. The one or more additional inputs may additionally or alternatively include one or more user inputs received during the performance of the medical procedure (e.g., input text, audio captured, etc.).

In an example, second machine learning model 406 may be a medical image captioning model. Medical image captioning models may use a variety of approaches to solve the problem of generating a text describing objects depicted within a medical image. Some example approaches include, but are not limited to, template-based approaches, retrieval-based approaches, generative models (e.g., encoder-decoder approaches), various hybrid techniques, and/or combinations thereof. The different approaches may impact the training process for training second machine learning model 406. For example, a training process different from training process 500 described above may be used based on the particular models to be trained.

Process 400 may include additional machine learning models, or first machine learning model 404 and/or second machine learning model 406 may be configured to perform additional or alternative functionalities. For example, process 400 may include a machine learning model trained to merge auto-generated text (e.g., data representing text 408 output by second machine learning model 406) with user-provided text (e.g., data representing user-provided text generated based on an utterance captured by an audio sensor). The intelligent merging of the auto-generated text and the user-provided text may be based on a medical profile associated with a user (e.g., a user who performed the medical procedure), which may store rules for respectively weighting auto-generated text and user-provided text. For example, the rules may indicate that additional weight may be assigned to the user-provided text when creating text 408 if the auto-generated text and the user-provided text differ. In another example, an additional machine learning model may be included in process 400 for adding graphics and/or other visual descriptors to image 402 based on the detected medical objects and/or the generated text (e.g., text 408). The additional (or alternative) machine learning models are described in greater detail below.

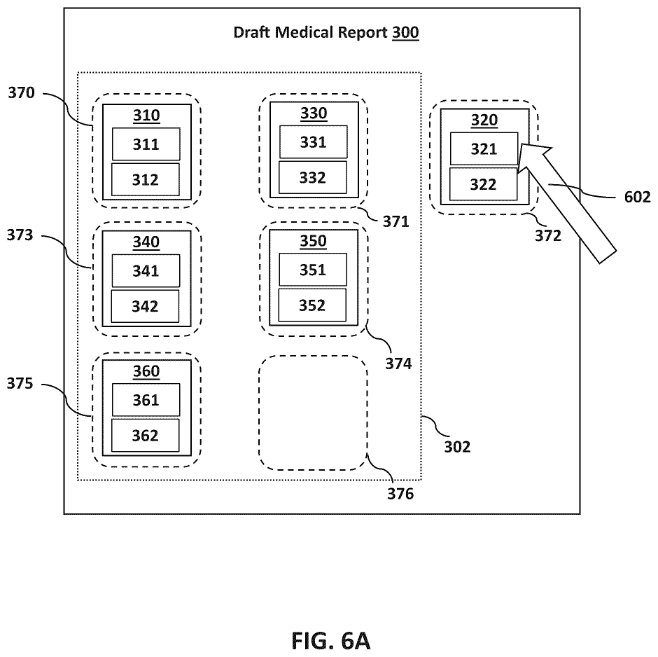

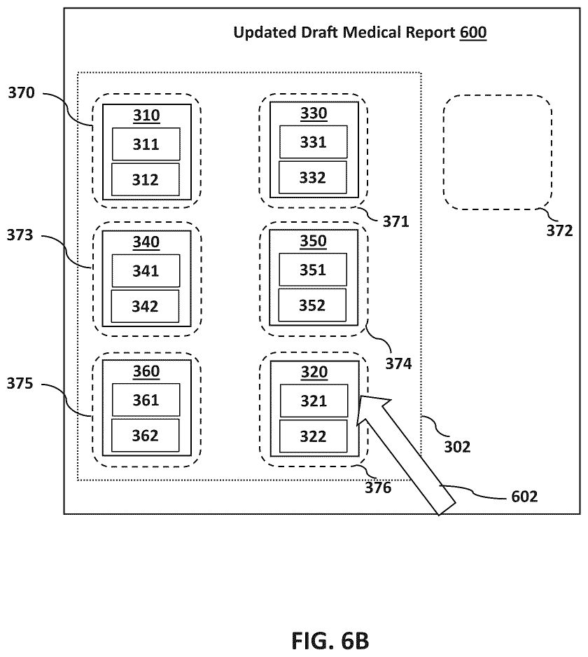

FIGS. 6A-6B illustrate examples of draft medical report 300 and an updated draft medical report 600 including a user selection of suggested content according to some aspects. As mentioned above, draft medical report 300 and/or updated draft medical report 600 may be presented to a user via client device 130. The user may correspond to a user that performed and/or assisted in performing a medical procedure described by draft medical report 300 and updated draft medical report 600. The user may select suggested content 320 for inclusion in draft medical report 300.

Client device 130 may detect a user selection 602 using a touchscreen, motion sensor, microphone, or other input device. As an example, user selection 602 may comprise a swipe, click, and/or drag operation detected via a touchscreen of client device 130. User selection 602 may indicate that suggested content 320 has been selected for inclusion in draft medical report 300. User selection 602 may also indicate a location within area 302 of draft medical report 300 that the suggested content 320 is to be displayed. For example, user selection 602 may indicate that suggested content 320 should be placed in a new region 376 in area 302. If user selection 602 comprises a drag operation, suggested content 320 (including auto-generated image 321 and/or auto-generated text 322) may be selected and dragged from region 372 and into region 376 for inclusion within draft medical report 300. Updated draft medical report 600, including suggested content 320 within area 302, may be stored in medical report database 174 (shown in FIG. 1B).

After suggested content 320 has been added to area 302, region 372 may display additional suggested content in region 372 or may not display any additional content. The suggested content may include a suggested image, suggested text describing the suggested image, and/or other suggested content that the machine learning models described above identified as depicting one or more image descriptors associated with a phase of the medical procedure. For example, suggested content that may be presented within region 372 (e.g., after suggested content 320 has been moved to area 302) may include auto-generated text describing an image and/or user-provided text describing an image. For example, medical report generation subsystem 110 may be configured to present the auto-generated text or the user-provided text based on which has a heavier weight (as described above). Medical report generation subsystem 110 may compare the auto-generated text and the user-provided text and present the auto-generated text and the user-provided text together in region 372 (or another suggested content region not explicitly illustrated in FIGS. 6A-6B). Medical report generation subsystem 110 may resolve differences in the auto-generated text and the user-provided text by selecting one or more terms from the text (e.g., auto-generated text or user-provided text) having the greater weight. The weights associated with each of the auto-generated text and the user-provided text may be presented in draft medical report 300, which may allow a user to see why certain terms were used to describe an image. This may also allow the user to select alternative terms, such as the terms from the lower-weighted text. In some examples, a tokenization process may be performed to tokenize the auto-generated text and the user-provided text. The tokenized auto-generated text and the tokenized user-provided text may then be compared to determine similarities and differences. The similarities and differences may be analyzed based on weights assigned to each of the auto-generated text and user-provided text to formulate updated auto-generated text. For example, different weights may be assigned to the terms of the auto-generated text and the user-provided text that may indicate whether the terms originating from the user-provided text or from the auto-generated text should be included in the updated auto-generated text. Differences between the user-provided text and the auto-generated text may be resolved using the weights.

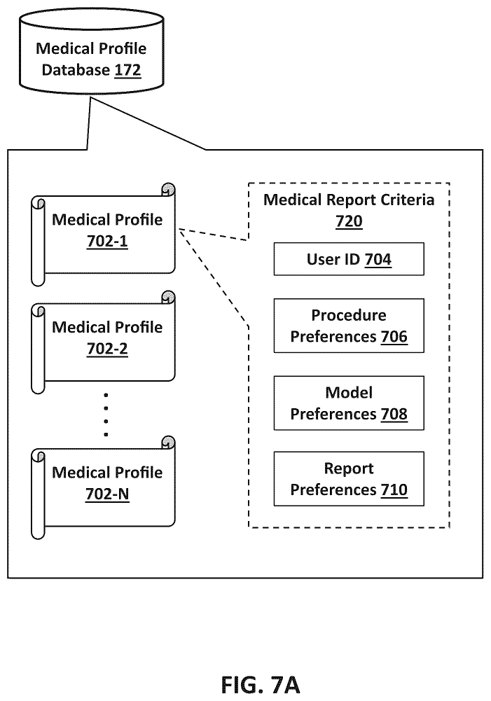

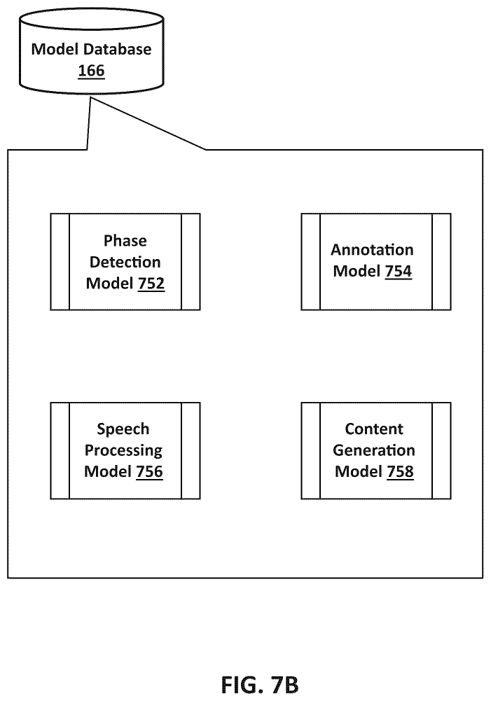

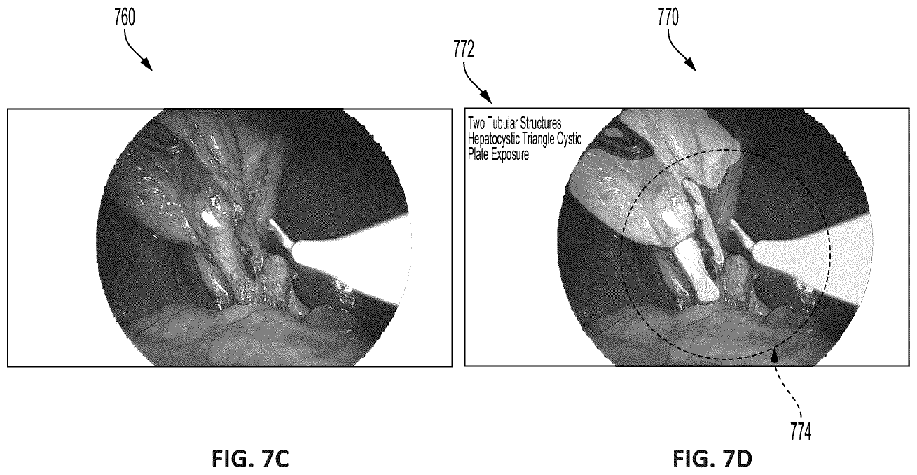

Information may be provided from updated draft medical report 600 to model training subsystem 114 indicating suggested content that has been selected by the user. Model training subsystem 114 may use the information to tune one or more hyperparameters of a machine learning model for generating future draft medical reports. For example, model training subsystem 114 may adjust weights and/or biases of a machine learning model based on suggested content 320 being added to draft medical report 300 to obtain updated draft medical report 600. Additionally or alternatively, medical report preferences of the user (e.g., report preferences 710 of FIG. 7A) may be updated based on the user selection, as will be described in greater detail below. This may further improve the efficiency of the medical report generation process by continually learning the types of content the user prefers to include in the draft medical report, thereby enabling future medical reports to be automatically formed based on the user's updated preferences.

An organization/arrangement of draft medical report 300 and/or updated draft medical report 600 may also be updated by medical report generation subsystem 110 based on user selection 602. For example, content (e.g., images, text, video, etc.) may be removed or moved to a different region of draft medical report 300 and/or updated draft medical report 600. The re-arrangement may be based on various factors, such as a temporal order of the images, an importance assigned to each of the images, user preference for the images, a quality of the images, other factors, or combinations thereof. For example, medical report generation subsystem 110 may re-arrange draft medical report 300 such that auto-generated content 310, 320, 330, 340, 350, and 360 are presented in chronological order. As another example, medical report generation subsystem 110 may re-arrange draft medical report 300 to obtain updated draft medical report 600 such that content associated with certain phases (e.g., earlier phases) of the medical procedure is presented prior to content associated with other phases (e.g., later phases) of the medical procedure. In another example, medical report generation subsystem 110 may re-arrange draft medical report 300 based on medical report criteria stored in a medical profile (stored in medical profile database 172 shown in FIG. 1B) of the user associated with the medical procedure (e.g., a surgeon performing a medical procedure, a medical professional who assisted in the medical procedure, etc.).

A user (e.g., surgeon, medical professional, etc.) may want to include content in the draft medical report 300 in addition to or instead of the auto-generated content. For example, the auto-generated content may not include all of the information that the user would like to include in draft medical report 300. In this case, the user may manually select additional images from the surgical video feed to add to draft medical report 300. However, combing through all of the frames from the surgical video feed may be time consuming. To make this process more efficient, medical report generation subsystem 110 may be configured to auto-generate time codes for the surgical video feed. The time codes can correspond to the detected surgical phases and any anomalous or otherwise significant medical events that occurred during the medical procedure. The corresponding auto-generated text may also be linked to the time codes. This can allow the user to quickly navigate to frames in the surgical video feed associated with the auto-generated text.

The user may select a time code and may be presented with content associated with the selected time code. For example, the user can select a time code and be provided with a video fragment including frames from the surgical video feed temporally associated with the selected time code. The user may be able to select one or more of the frames forming the video segment to be added to the draft medical report. For example, the user may manually insert the selected frames into draft medical report 300. Based on the selection, medical report generation subsystem 110 may analyze the selected frames, as well as other frames associated with the selected time segment, to determine whether any of the selected frames represent a medical event that should have been documented. If so, medical report generation subsystem 110 may include one or more of those frames as suggested content.

In addition to or instead of time codes, medical report generation subsystem 110 may be configured to generate a search index of frames from the surgical video feed. The search index may link frames with keywords, which may also be associated with medical events. The search index may further link frames with phases of the medical procedure. Medical report generation subsystem 110 may allow a user to input a search query and may retrieve frames relevant to the search query. For example, the user may input a free-form query (e.g., “take me to the critical view of safety stage”), and medical report generation subsystem 110 may automatically search the index and match the free-form query to one or more frames. The frames may describe medical events semantically related to the terms included in the query. Medical report generation subsystem 110 may be configured to retrieve the semantically related frames and present a video segment formed of the semantically related frames to the user.

FIG. 7A illustrates example medical profiles stored in medical profile database 172, according to some aspects. For instance, medical profile database 172 may include medical profiles 702-1, 702-2, . . . , 702-N, which collectively may be referred to as medical profiles 702 or individually as medical profile 702. Medical profiles 702 may each be associated with a particular user. For example, each of medical profiles 702 may be associated with a user (e.g., a surgeon, medical professional, etc.) and may include information about and/or preferences of that user. Medical profile database 172 may also include a base medical profile that may be associated with a new user. The base medical profile may be generated using one or more medical profiles of experienced users (e.g., a user with X number of years of experience, a user that has previously performed X medical procedures, etc.).

As mentioned above, medical profile 702 may be provided to one or more machine learning models used to analyze images of the medical procedure. The machine learning models may determine whether the images include image descriptors associated with one or more phases of the medical procedure. Those that the machine learning models determine include image descriptors may be selected for possible inclusion in the draft medical report (e.g., draft medical report 300).

As mentioned above, each medical profile 702 may include medical report criteria 720 for the corresponding user. Medical report criteria 720 may include information and/or preferences associated with the corresponding user. For example, medical report criteria 720 may include a user identifier (ID) 704, procedure preferences 706, model preferences 708, report preferences 710, other preferences of a user, other information relating to the user, and/or combinations thereof. User ID 704 may include identification information that can be used to identify a user associated with a given medical profile. For example, user ID 704 may refer to a user's name, title, employee number, login information (e.g., username, password, etc.), email address, or other information.

Procedure preferences 706 may include preferences of the user with respect to one or more medical procedures that the user performs. For example, a user, such as a surgeon, may perform one or more medical procedures, and procedure preferences 706 may store information associated with medical events that the user prefers to document in a medical report for those medical procedures. For example, the medical events indicated in procedure preferences 706 may include detecting particular objects (e.g., anatomical structures) that the user prefers to document (e.g., in one or more images). For example, the user may prefer to document one or more images corresponding to a beginning of a phase of the medical procedure. Procedure preferences 706 may include indications of objects typically depicted within images captured during a given phase of the medical procedure. Thus, during performances of the medical procedure, images depicting those objects may be captured. As another example, the user may prefer to document a “before” image and an “after” image during the medical procedure. Therefore, procedure preferences 706 may include image descriptors of a “before” image and an “after” image such that, during performances of the medical procedure, images depicting those image descriptors may be captured. Procedure preferences 706 may include time windows during which particular medical events are expected to occur during a medical procedure. For example, with reference to FIG. 2, each of time windows T1-T6 may represent a range of times that an image corresponding to a specific medical event was captured during prior performances of the medical procedure by that user.

Model preferences 708 may include indications of which machine learning models the user prefers to use to analyze images/video of the medical procedure. Model preferences 708 may instead or additionally include settings for hyperparameters of machine learning models (e.g., stored in model database 166) based on prior uses of those machine learning models to analyze images/video and/or generate draft medical reports. For example, model preferences 708 may include a machine learning model and/or settings for a machine learning model to be used when detecting objects associated with a medical procedure.

Report preferences 710 may include indications of the user's medical report preferences for draft medical reports. For example, report preferences 710 may include and/or indicate templates that the user prefers to use to create draft medical reports (e.g., draft medical report 300). The templates may indicate the locations at which the user prefers to display content within draft medical report 300, the type of content to include, the amount of content to include, weights used in including user-provided comments (e.g., weights used to merge user-provided comments with auto-generated text), or other medical report preferences. Report preferences 710 may be updated based on one or more user selections made with respect to the draft medical report. For example, as described above at least with respect to FIGS. 6A-6B, a user selection of auto-generated image 321 may result in an update to report preferences 710 such that future medical reports for a given medical procedure may include images similar to auto-generated image 321.

FIG. 7B illustrates example machine learning models stored in model database 166, according to some aspects. Model database 166 may store untrained and/or trained machine learning models. Some example machine learning models that may be stored in model database 166 include a phase detection model 752, an annotation model 754, a speech processing model 756, a content generation model 758, and/or other machine learning models.

Phase detection model 752 may be trained to determine a particular phase of a medical procedure based on images captured during the medical procedure. Phase detection model 752 may be trained using a process that is the same or similar to process 500. Phase detection model 752 may determine phases of a given medical procedure based on image descriptors detected within images of the medical procedure. As mentioned above, the image descriptors may include objects, environmental factors, and/or contextual information associated with one or more phases of the medical procedure. For example, the image descriptors may indicate a particular anatomical structure whose presence within an image may indicate that the medical procedure has entered a first phase. As the medical procedure progresses, different image descriptors associated with different phases of the medical procedure may be detectable by phase detection model 752. Images depicting the image descriptors may be selected (e.g., based on procedure preferences 706) for possible inclusion in draft medical report 300 (shown in FIG. 3).

Annotation model 754 may be trained to annotate images for draft medical report 300. Annotation model 754 may be trained using a process that is the same or similar to process 500. The annotations may include text annotations, graphics, video, audio, and/or other annotations. For example, as seen with respect to FIGS. 7C and 7D, original image 760 may be updated to include annotations 772 and/or 774, as seen in annotated image 770. Annotation 772 may include information related to the medical procedure depicted by annotated image 770. Annotation 774 may indicate a particular (e.g., important) aspect of annotated image 770. Annotation model 754 may detect (e.g., directly and/or from phase detection model 752) medical objects depicted within an image. Based on the detected medical objects, annotation model 754 may determine a type of annotation to make (if any), a location for the annotation, etc. The annotations may be determined based on the medical profile of the user performing the medical procedure. For example, medical profile 702 may be provided to annotation model 754 to auto-generate annotations (e.g., annotations 772, 774). As mentioned above, medical profile 702 may include report preferences 710, which may indicate preferences of the user for annotating images.

In some aspects, graphic tools may be provided to the user for adding annotations to content (e.g., auto-generated images). For example, a set of graphic tools may be rendered on a user interface displaying a draft medical report (e.g., on a display of client device 130 of FIG. 1B) to allow the user to manually annotate one or more auto-generated images. The graphic tools may include various image editing tools, such as textboxes, arrows, free-hand strokes, shapes, etc. Additionally, one or more advanced image enhancement tools may be provided to the user within a user interface displaying the draft medical report. These advanced image enhancement tools may be based on traditional computer vision analysis and may include, for example, contrast enhancement, histogram equalization, color maps, etc. The user (e.g., surgeon, medical professional, etc.) may use a combination of manual annotations and advanced enhancement tools. For example, the user may manually outline a portion of the image and apply a manipulation to the corresponding portion (e.g., zoom-in, enhance, color, etc.).

Returning to FIG. 7B, speech processing model 756 may be configured to receive audio data representing sounds detected, or otherwise captured, by one or more audio sensors (e.g., microphones 16 shown in FIG. 1A) during the medical procedure, before the medical procedure, and/or after the medical procedure. For example, during the medical procedure, the user (e.g., surgeon medical professional, etc.) may speak an utterance regarding an aspect of the medical procedure. Speech processing model 756 may be configured to receive audio data of the audio, wherein the audio represents the utterance. Speech processing model 756 may be configured to generate text representing the audio (e.g., speech-to-text) and/or determine an intent of the utterance based on the text. For example, speech processing model 756 may employ natural language processing to determine the intent of the utterance using lexical and semantic analyses. Speech processing model 756 may further be configured to generate text (e.g., prose) describing an image. For example, speech processing model 756 may generate text and/or retrieve pre-generated text describing objects detected within a captured image. Speech processing model 756 may use the generated text and/or pre-generated text to form text for inclusion in the draft medical report. Speech processing model 756 may be configured to merge text (e.g., auto-generated text and user-provided text) based on one or more weighting rules. With reference to FIGS. 6A-6B, auto-generated text 312, 322, 332, 342, 352, and 362 may represent example text generated by speech processing model 756. Speech processing model 756 may be configured to begin generating text representing the audio in response to a triggering event indicating that text representing the audio should be generated. The triggering event may be, for example, a wake word spoken by a user. The speech processing model 756 may be configured to stop generating text representing the audio in response to a triggering event indicating that the text should stop being generated. The triggering event indicating that the text should stop being generated could be, for example, a user spoken command to stop generating text representing the audio or user silence for a predetermined time. Speech processing model 756 may be configured to start and/or stop generating text representing the audio based on ranges of times within which certain medical events associated with the medical procedure are expected to occur, such as described above with respect to time windows T1-T6 illustrated in FIG. 2. For example, speech processing model 756 may automatically start generating text representing the audio at the start t1 of time window T4 of FIG. 2 and may automatically stop generating text representing the audio at the end t2 of time window T4. Additionally, or alternatively, speech processing model 756 may automatically start generating text representing the audio at the start of a procedure, such as at the start of time window T1 of FIG. 2 and may automatically stop generating text representing the audio at the end of the procedure, such as at the end of time window T6 of FIG. 2.

Content generation model 758 may be trained to generate a draft medical report (e.g., draft medical report 300), suggested content (e.g., suggested content 320), and/or a user interface for presenting the draft medical report and/or the suggested content. For example, content generation model 758 may be configured to generate draft medical reports based on preferences of the user, images captured, speech detected, annotations added, other factors, and/or combinations thereof. Content generation model 758 may determine which images (if any) are to be included as suggested images. For example, with reference to FIG. 2, content generation model 758 may identify an image depicting a medical object expected to be visible during time window T1 but instead was detected during time window T3. This image may be included in draft medical report 300 as a suggested image (e.g., part of the suggested content, such as suggested content 320). Content generation model 758 may determine that this image should be included as suggested content (e.g., suggested content 320) for the user to potentially include in draft medical report 300 (depicted in FIGS. 3 and 6A). Content generation model 758 may be configured to identify key information in an image and filter out unwanted details.

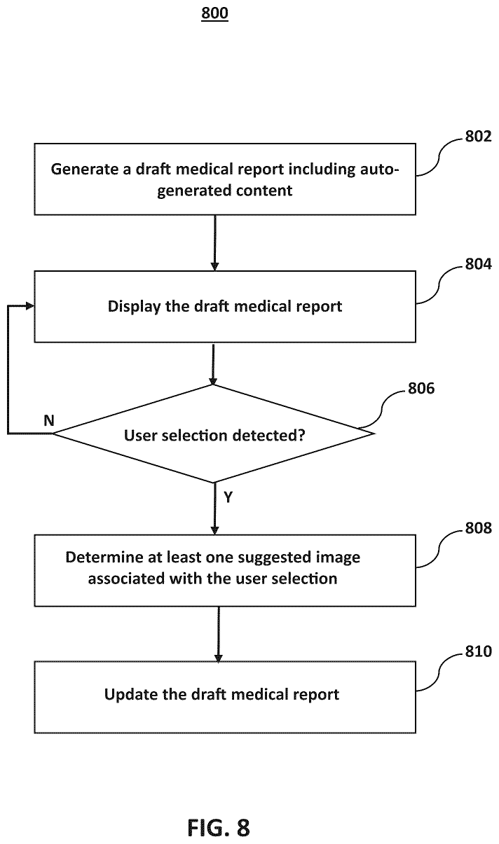

FIG. 8 illustrates a flowchart of an example method 800 for generating and updating a draft medical report, according to some aspects. Method 800 may begin at step 802. At step 802, a draft medical report may be generated including auto-generated content. A draft medical report may be created based on content captured during a medical procedure (e.g., images and/or videos), information associated with the medical procedure (e.g., pre-op/post-op images, test results, etc.), preferences of a user (e.g., a surgeon, medical professional, etc.) associated with the medical procedure, and/or other medical report criteria (described above at least with respect to FIG. 7A). For example, draft medical report 300 may be created including auto-generated content 310, 320, 330, 340, 350, and 360, illustrated at least in FIG. 3. Auto-generated content 310, 320, 330, 340, 350, 360 may include images 311, 331, 341, 351, and 361, as well as text 312, 332, 342, 352, and 362. Some of auto-generated content 310, 320, 330, 340, 350, and 360 may be suggested content, such as, for example, suggested content 320. Suggested content 320 may include auto-generated image 321 and auto-generated text 322. Auto-generated content 310, 320, 330, 340, 350, and 360 may be selected for possible inclusion in draft medical report 300 based on image descriptors detected within those images. The image descriptors may indicate classifications for the images. The classifications may be associated with medical events of the medical procedure.

As mentioned above, an input video feed may be received by medical report generation subsystem 110 from medical device 120 (e.g., an endoscope). The input video feed may depict a medical procedure being performed by a user (e.g., surgeon, medical professional, etc.). Medical report generation subsystem 110 may analyze frames of the input video feed (e.g., image 402) to determine whether any of the frames depict image descriptors associated with medical events to be documented in a draft medical report. One or more of the medical events to be documented may be determined based on a medical profile of the user, which can include report preferences 710 (e.g., illustrated in FIG. 7A). If image descriptors associated with the medical events are detected within the input video feed, medical report generation subsystem 110 may include the corresponding frames (e.g., sequenced images forming the input video feed) in the draft medical report (e.g., draft medical report 300 in FIG. 3).

Medical report generation subsystem 110 may also suggest auto-generated content, such as suggested content 320, to the user for inclusion in draft medical report 300. Suggested content 320 may be presented to the user with auto-generated content 310, 330, 340, 350, and 360 of draft medical report 300 of FIG. 3. Suggested content 320 may include images, video, and/or other content that depict medical events in the medical procedure. For example, suggested content 320 (e.g., captured at time 220) may depict an unexpected event or an action that may be important to document in draft medical report 300. For example, an unexpected event or action may be an action performed by a user in a given medical procedure that is not typically performed during that medical procedure. Medical report generation subsystem 110 may capture content depicting the unexpected event, and may provide the content as suggested content for draft medical report 300.

Medical report generation subsystem 110 may also include content captured in response to a user selection in a draft medical report. For example, a user may determine that a particular aspect of the medical procedure is to be documented in draft medical report 300. The user may invoke an image capture option to manually capture an image of that aspect. The captured content may be included in draft medical report 300.

Optionally, medical report generation subsystem 110 starts and/or stops analysis of frames of the input video feed for possible inclusion in a medical report during step 802 based whether the input video feed indicates that the associated camera (e.g., camera head 140 coupled to endoscope 142) is directed at a surgical site. For example, the medical report generation subsystem 110 may not start analyzing frames of the input video feed for possible inclusion in a medical report until the video is capturing a surgical site (e.g., the endoscope 142 is inserted inside the patient) and/or may stop analyzing frames of the input video feed for possible inclusion in the medical report when the video is no longer capturing the surgical site. This may improve accuracy of the medical report by ensuring that portions of the video feed that do not depict the surgical site (which may not be usable for the medical report) will not be included in the medical report. Further, this may prevent the unintentional inclusion of images containing protected health information (PHI) or personally identifiable information (PII), which may be captured in the input video feed (e.g., a patient's face or other identifying features) prior to the camera being directed at the surgical site (e.g., when the endoscope 142 is outside the body). Analysis of frames of the input video feed by medical report generation subsystem 110 may be stopped and started multiple times, such as when a user withdraws the endoscope 142 from within the body for cleaning (stop image analysis) and reinserts the endoscope into the body (start image analysis). One or more machine learning models may be trained to detect whether the input video feed is capturing a surgical site or not. For example, phase detection model 752 may be trained to detect when endoscope 142 is inside the body or not. Additionally or alternatively to start and/or stop analysis of frames of the input video feed for possible inclusion in a medical report, medical report generation subsystem 110 may start and/or stop storage of frames based whether the input video feed indicates that the associated camera (e.g., camera head 140 coupled to endoscope 142) is capturing a surgical site. This may prevent the unintentional retention of images containing protected health information (PHI) or personally identifiable information (PII).

At step 804, the draft medical report may be displayed to the user. Draft medical report 300 may be displayed via a graphical user interface (GUI) rendered on medical device 120, client device 130, and/or another device. For example, additional monitor 14 within medical environment 10 in FIG. 1A may render a GUI displaying draft medical report 300. Draft medical report 300 may include auto-generated content, such as auto-generated content 310, 320, 330, 340, 350, and 360. The auto-generated content may include suggested content, for example, suggested content 320, which may include auto-generated image 321 and auto-generated text 322.

At step 806, a determination may be made as to whether a user selection has been detected. A user selection may refer to an input detected by computing system 102, medical device 120, client device 130, and/or another device within system 100 in FIG. 1B. In an example, a user selection detected on a device other than computing system 102 may cause a notification to be generated and sent to computing system 102 indicating the selection and any additional pertinent data. The user selection may indicate suggested content that the user wants to include in the draft medical report. Alternatively or additionally, the user selection may select auto-generated content the user seeks to remove from the draft medical report. Still further, the user selection may comprise an indication to re-structure or re-arrange the medical report and the content included therein. The user selection may be a touch-sensitive input (e.g., detected via a touchscreen of medical device 120, client device 130, or another device displaying draft medical report 300) and/or a non-touch-sensitive input, such as mouse clicks, stylus interactions, etc. The user selection may instead or additionally be detected via a voice command and/or a gesture detected by an image sensor and/or motion sensor, respectively. At step 806, if it is determined that no user selection has been detected, method 800 may return to step 804 and continue to display the draft medical report.

At step 806, if it is determined that a user selection has been detected, then method 800 may proceed to step 808. At step 808, at least one suggested image associated with the user selection may be determined. More generally, the user selection may relate to some or all of the suggested content. For example, with reference to FIGS. 6A and 6B, user selection 602 may select suggested content 320 to include in draft medical report 300.

At step 810, the draft medical report may be updated. For example, medical report generation subsystem 110 may be configured to update draft medical report 300 based on user selection 602. User selection 602 may include an action to cause the selected content (e.g., suggested content 320) to be included in draft medical report 300. As described above with respect to FIGS. 6A and 6B, user selection 602 may comprise moving suggested content 320 from region 372 to region 376 within area 302. The updated draft medical report may be presented to the user.

Updated draft medical report 600, draft medical report 300, auto-generated content 310, 320, 330, 340, 350, and 360, or other data, may be stored in medical report database 174. Additionally or alternatively, auto-generated content 310, 320, 330, 340, 350, and 360, draft medical report 300, and/or updated draft medical report 600 may be stored in image database 162. The user selections may be stored in medical report database 174 and may be provided to model database 166 and/or model training subsystem 114 to update the machine learning model(s) used to generate the draft medical report. For example, the machine learning models may be updated based on the user's preferences.

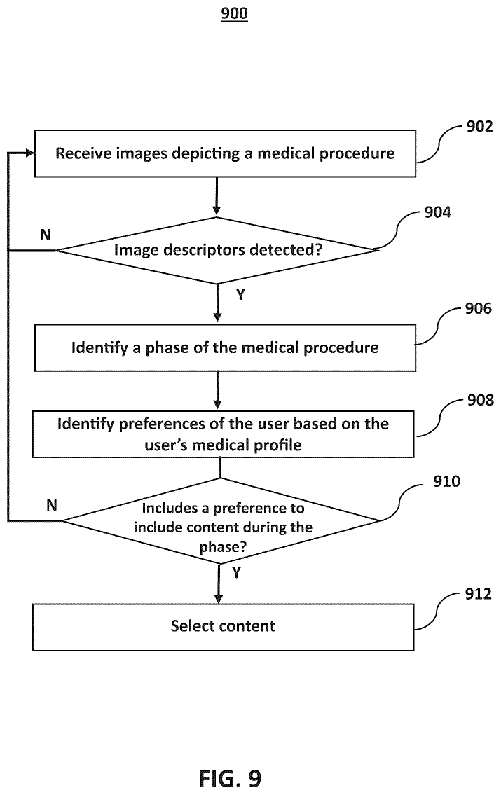

FIG. 9 illustrates a flowchart of an example method 900 for determining whether an image captured during a medical procedure is to be selected for inclusion in a draft medical report, according to some aspects. Method 900 may begin at step 902. At step 902, one or more images depicting a medical procedure may be received. In some instances, medical procedures may employ a medical device including one or more image sensors. For example, an endoscope may be used to assist in performing certain minimally invasive medical procedures. The image sensors, such as those of an endoscope, may provide a stream of images and/or video of the medical procedure to a computing device, which may analyze the images and perform one or more actions based on the images. The images may depict external views of a patient, internal (e.g., anatomical) structures of a patient, and/or other aspects of a medical procedure.

At step 904, a determination may be made as to whether any image descriptors have been detected within the received images. The image descriptors may include one or more objects, environmental factors, and/or contextual information associated with phases of the medical procedure. The received images may be analyzed by one or more machine learning models trained to detect image descriptors. Content including image descriptors representing some or all of these phases may be included within a draft medical report, such as draft medical report 300 of FIG. 3. The computer vision model (e.g., first machine learning model 404 in FIG. 4) may classify content based on the image descriptors detected within the content. For example, images may be classified as depicting one or more image descriptors or not depicting any image descriptors. If one or more image descriptors are detected in an image, the computer vision model may be configured to classify the image into one or more predefined categories associated with potential detected image descriptors. For instance, at step 904, if it is determined that the received images do not include any image descriptors associated with the medical procedure being performed, then method 900 may return to step 902 where additional images may be received and analyzed. However, at step 904, if one or more medical objects are detected, method 900 may proceed to step 906.

At step 906, a phase of the medical procedure may be identified. The medical procedure may include one or more phases (e.g., preoperative phase, intraoperative phase, postoperative phase, etc.) where certain medical events occur. The medical events may be detectable based on image descriptors in the images captured during the medical procedure. For example, a detected image descriptor may be associated with a given medical event, and that medical event can be used to determine the phase of the medical procedure. For example, detecting a first anatomical structure may indicate that a first phase of the medical procedure has begun. In another example, detecting a second anatomical structure may indicate that a second phase of the medical procedure has ended. In another example, a previously detected image descriptor that is no longer present within the received images may indicate a transition from one phase of the medical procedure to another phase.

At step 908, one or more preferences of the user may be identified based on the user's medical profile. The identity of the user may be determined based on credentials of the user (e.g., an RFID tag, a device detected that is associated with the user, retinal scan, facial scan, fingerprinting, manual input, or other techniques). The user may alternatively or additionally be identified based on scheduling data associated with the user and/or the medical procedure. Upon determining the identity of the user, the medical profile of the user may be obtained. As mentioned above, medical profiles, such as medical profile 702, may include preferences related to the type of content to include in a draft medical report describing the medical procedure. For example, procedure preferences 706 illustrated in FIG. 7A may indicate which phases of a medical procedure the user prefers to include images of in medical reports. The phases of interest may be determined based on prior performances of the medical procedure. For example, in the instance a user has performed a particular medical procedure 10 times, and during each of those prior performances the user has captured an image of a given anatomical structure, then the preferences of the user stored in the medical profile may indicate that an image depicting the given anatomical structure should be captured during any subsequent performances of the medical procedure. The prior performances of a medical procedure may also be used to determine time windows when the anatomical structure is expected to be visible in the medical procedure. Continuing with the previous example, during each of the 10 prior performances, the anatomical structure may have been detected within a particular time window. Therefore, during the medical procedure, medical report generation subsystem 110 may indicate to the machine learning vision model(s) analyzing the images the times at which those anatomical structure may be detected.

At step 910, a determination may be made as to whether the preferences of the user include a preference to include content captured during the identified phase (e.g., identified in step 906) in a draft medical report. In other words, when certain image descriptors, such as medical objects, are detected, medical report generation subsystem 110 may determine whether to select content depicting the image descriptors for inclusion in the draft medical report based on user preferences. Detecting those medical objects may indicate that the medical procedure has entered a particular phase, and based on user preferences, it may be determined that content describing that particular phase are typically included in draft medical reports. If a preference related to the phase is not identified, method 900 may return to step 902, where additional images (e.g., from a surgical video feed) may continue to be received. However, at step 910, if it is determined based on preferences that the content is to be included in the draft medical report, method 900 may proceed to step 912 where the content may be selected. This captured content may represent an image, video, audio, text, and/or other content that has been extracted from the surgical video feed (e.g., one or more frames) and used in the draft medical report. The content from the video surgical feed may continually be analyzed, stored, and/or purged from memory.

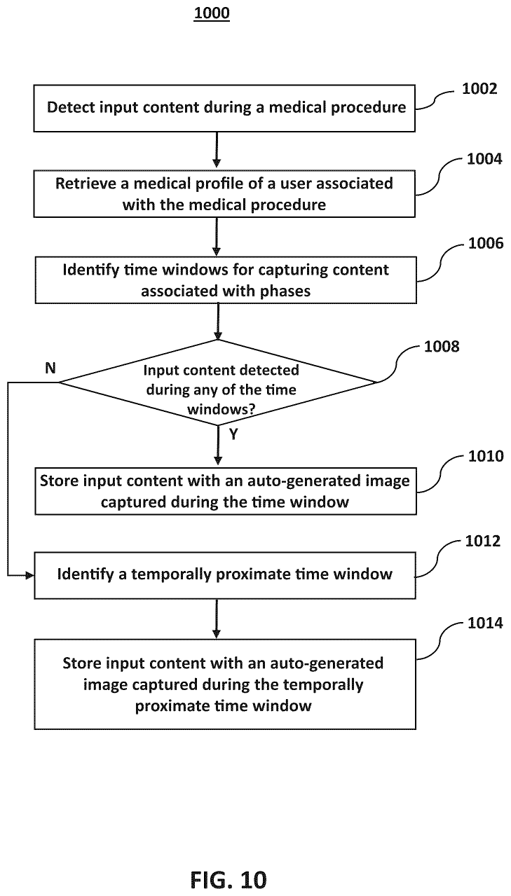

FIG. 10 illustrates a flowchart of an example method 1000 for associating content (e.g., input audio, text, etc.) inputted during the medical procedure with other content captured during the medical procedure (e.g., one or more captured images), according to some aspects. Method 1000 may begin at step 1002. At step 1002, input content, such as audio and/or text, may be detected during a medical procedure. For example, medical report generation subsystem 110 may receive data representing the input content. For example, input content may comprise audio detected from a user (e.g., surgeon, medical professional, etc.) speaking an utterance (e.g., describing a particular aspect of the medical procedure). Microphones 16 disposed within medical environment 10 may receive audio signals of the utterance and generate audio data representing audio of the utterance. Medical report generation subsystem 110 may receive the audio data of the audio from microphones 16. Medical report generation subsystem 110 may generate text representing the audio using one or more speech processing models (e.g., speech processing model 756 illustrated in FIG. 7B). Medical report generation subsystem 110 may perform semantic and lexical analysis to the text to determine what was uttered by the user in the audio.

In another example, input content may comprise gestures (hand gestures, eye movement, head motion, etc.) detected from a user (e.g., surgeon, medical professional, etc.). One or more cameras (e.g., room camera 146 or camera 152 in surgical light 154, etc.) disposed within medical environment 10 may capture videos or images of the gesture. Additionally or alternatively, one or more motion sensors disposed within medical environment 10 may detect the motion and generate data representing the motion. Medical report generation subsystem 110 may receive the video or image data of the gesture from the one or more cameras and/or the data representing the gesture from the one or more motion sensors. Medical report generation subsystem 110 may identify the gesture using one or more machine learning models. Different gestures may be associated with different meanings. For example, the user may make a gesture that indicates a first phase of the medical procedure has begun. In another example, a gesture may be associated with text to be included in a draft medical report.

At step 1004, a medical profile of a user associated with the medical procedure may be retrieved. For example, the medical profile may be retrieved from medical profile database 172, which may store medical profiles associated with a number of users. The user may be identified based on scheduling data associated with the user and/or the medical procedure. For example, scheduling data may indicate that a particular user (e.g., a surgeon, medical professional, etc.) is performing the medical procedure within the medical environment (e.g., medical environment 10 illustrated in FIG. 1A). Alternatively or additionally, the user may be identified based on log-in credentials (e.g., username, employee identifier, an RFID tag, a device detected that is associated with the user, retinal scan, facial scan, fingerprinting, manual input, or other techniques), activities being performed within the medical environment (e.g., the user is identified in the medical environment as holding an endoscope), and/or other identification techniques (e.g., facial recognition, voice recognition, etc.). The user may alternatively or additionally be identified based on scheduling data associated with the user and/or the medical procedure.

At step 1006, time windows for capturing content associated with phases of the medical procedure may be identified. For example, the medical profile may include preferences of the user, which may include time windows during which certain medical events are expected to occur. Different medical events may reflect different phases of the medical procedure. For example, one medical event (e.g., the detection of an anatomical structure) may indicate that a first phase of the medical procedure has begun, while another medical event (e.g., the detection of another anatomical structure, the absence of a previously visible anatomical structure, etc.) may indicate that a second phase of the medical procedure has ended.

At step 1008, a determination may be made as to whether the input content (e.g., input audio, text, and/or gesture) was detected during one of the time windows. If, at step 1008, it is determined that the input content was detected during one of the time windows, method 1000 may proceed to step 1010. At step 1010, the input content may be stored in association with content captured during the corresponding time window. For example, if audio/text is detected at time 210 in FIG. 2 (during time window T1), medical report generation subsystem 110 may store the audio/text in association with auto-generated content 310 captured at time 210. In another example, if a user gesture is detected at time 210 in FIG. 2 (during time window T1), medical report generation subsystem 110 may store the gesture or meaning of the gesture in association with auto-generated content 310 captured at time 210.

However, if it is determined that the input content was not captured during one of the identified time windows, method 1000 may bypass step 1010 and proceed to step 1012. At step 1012, a time window may be identified that is temporally proximate to the input content being detected. For example, with reference to FIG. 2, audio/text may be detected at time 270. Time 270 may be after time window T2 ends, but before time window T3 starts. Medical report generation subsystem 110 may determine whether the audio/text detected at time 270 should be associated with content captured during time window T2 or time window T3. Medical report generation subsystem 110 may determine a first amount of time that has elapsed from an end of time window T2 to time 270 and a second amount of time that has elapsed from time 270 until a beginning of time window T2. The amount of time that is determined to be smaller may indicate the time window to associate with the input content detected at time 270. The amount of time used to determine the temporally proximate time window may be based on any time point within a given time window, as long as consistency is used when computing the time differences. For example, a time point in the middle of time windows T2 and T3 may be used, and an amount of time between the middle of time window T2 and time 270 may be compared to time 270 and the middle of time window T3.

At step 1014, the input content may be stored as user-provided text for an image captured during the identified temporally proximate time window. For example, if the amount of time between the end of time window T2 and time 270 is smaller than the amount of time between time 270 and the start of time window T3, then the input content detected at time 270 may be stored as user-provided text for content captured during time window T2.

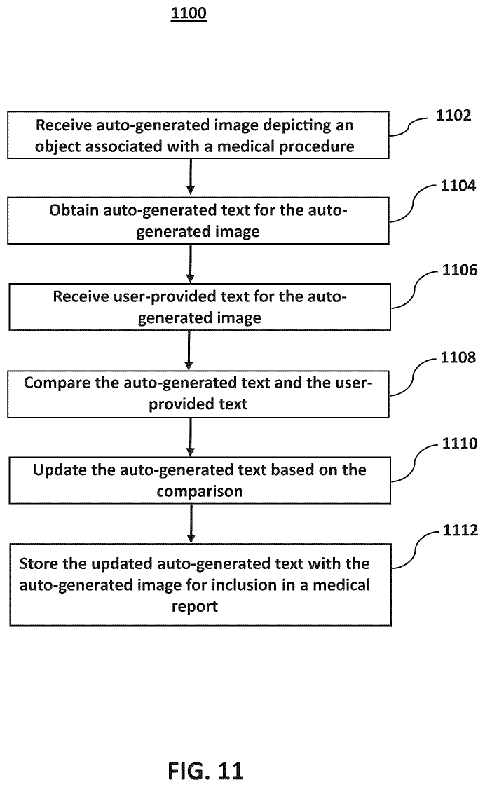

FIG. 11 illustrates a flowchart of an example method 1100 for intelligently merging auto-generated text and user-provided text, according to some aspects. Method 1100 may begin at step 1102. At step 1102, an auto-generated image depicting an image descriptor associated with a medical procedure may be received. For example, medical report generation subsystem 110 may receive an image depicting an anatomical structure viewable during a medical procedure. The image may be received from medical device 120 (e.g., an endoscope).

At step 1104, auto-generated text for the auto-generated image may be obtained. The auto-generated text may be generated by a machine learning model trained to generate text describing an image input to the machine learning model. For example, an image may be provided to a machine learning model trained to generate text describing an image descriptor detected therein. The machine learning model may generate the auto-generated text based on the detected image descriptor. For example, with reference to FIG. 4, second machine learning model 406 may generate text 408 describing image 402.

Additionally, one or more machine learning models may be configured to annotate and/or update the auto-generated content. In some examples, rather than two machine learning models (e.g., first machine learning model 404 and second machine learning model 406), image 402 may be provided as input into a single machine learning model that detects image descriptors present within image 402 and generates text 408 describing the detected objects. The auto-generated text may be generated using a medical lexicon. The medical lexicon may be created based on previously generated text (e.g., “pre-generated text”). The pre-generated text may describe image descriptors (e.g., objects, environmental factors, contextual information) associated with previously captured images from prior performances of the medical procedure.

At step 1106, user-provided text may be received as an input. The user-provided text may be provided as input by a user associated with the medical procedure, such as a surgeon performing the medical procedure or other personnel in the operating room. The input may be received via an input device (e.g., a keyboard, touchpad, touchscreen, or other device). If there is an audio input, the audio input may be received via an audio sensor, such as a microphone. For example, a user may speak an utterance and a microphone of client device 130 and/or medical device 120 (e.g., microphone 16 shown in FIG. 1B) may detect the utterance. The utterance may be spoken during the medical procedure, prior to the medical procedure, and/or after the medical procedure. If there is a gesture input, the gesture input may be received via an image sensor, such as one or more cameras disposed within a medical environment, or a motion sensor. For example, a user may make a gesture and one or more cameras disposed in medical environment 10 (e.g., room camera 146 and/or camera 152 in surgical light 154 shown in FIG. 1A) may detect the gesture. The gesture may be made during the medical procedure, prior to the medical procedure, and/or after the medical procedure. Medical report generation subsystem 110 may be configured to associate content (e.g., the utterance or gesture) with other content (e.g., images, text, audio, etc.) captured during the corresponding time window. Medical report generation subsystem 110 may be configured to obtain auto-generated text of the audio content captured during the corresponding time window. For example, text representing the utterance may be generated using one or more speech processing models, such as speech processing model 756 illustrated in FIG. 7B. Medical report generation subsystem 110 may be configured to obtain auto-generated text of the gesture captured during the corresponding time window. For example, the meaning of a gesture may be identified using a computer vision model, and text describing the meaning of the gesture may be generated using a machine learning model.

The user-provided text may be received during a time window different from those associated with phases of the medical procedure. For example, as mentioned above, microphone 16 within medical environment 10 in FIG. 1A may detect audio signals corresponding to an utterance spoken by a user within medical environment 10. The microphone may be triggered to begin capturing sounds continually or in response to a trigger being detected (e.g., an input mechanism being invoked, a wake word being uttered, etc.). Medical report generation subsystem 110 may be configured to determine the phase of the medical procedure related to the user-provided text. For example, with reference to FIG. 2, audio data representing audio of an utterance detected at time 270, which may be between an end of time window T2 and a start of time window T3, may be associated with suggested content 320 captured at time 220 in time window T2 or auto-generated content 330 captured at time 230 in time window T3. Medical report generation subsystem 110 may determine whether to associate the user-provided text with suggested content 320 or auto-generated content 330. A timestamp indicating a time that the user-provided text was detected may be compared to the end of the first time window (e.g., T2) and the start of the second time window (e.g., T3). For example, if the amount of time between the end of time window T2 and time 270 is smaller than the amount of time between time 270 and the start of time window T3, then the user-provided text may be associated with suggested content 320 captured during time window T2. As another example, if the amount of time between the end of time window T2 and time 270 is greater than the amount of time between time 270 and the start of time window T3, then the user-provided text may be associated with auto-generated content 330 captured during time window T3.

At step 1108, the auto-generated text may be compared to the user-provided text. A tokenization process may be performed to tokenize the auto-generated text and the user-provided text. The tokenized auto-generated text and the tokenized user-provided text may then be compared to determine similarities and differences. The similarities and differences may be analyzed based on weights assigned to each of the auto-generated text and user-provided text to formulate updated auto-generated text. For example, different weights may be assigned to the terms of the auto-generated text and the user-provided text that may indicate whether the terms originating from the user-provided text or from the auto-generated text should be included in the updated auto-generated text. Differences between the user-provided text and the auto-generated text may be resolved using the weights.

At step 1110, the auto-generated text may be updated based on the comparison between the auto-generated text and user-provided text. Alternatively, instead of updating the auto-generated text based on the comparison, the user-provided text may be updated based on the comparison. The comparison may indicate that certain terms included in the user-provided text were not included in the auto-generated text, thus, the auto-generated text may be updated to include one or more of the terms and/or phrases from the user-provided text. The medical profile of the user associated with the user-provided text may also be used to update the auto-generated text. The medical profile may include preferences, rules, weightings, etc., related to the manner in which the tokenized auto-generated text and user-provided text are to be merged. For example, the medical profile of the user may indicate that the user-provided text should be weighted greater than the auto-generated text. Therefore, if terms, phrases, utterances, etc. are included in the user-provided text and not the auto-generated text, then medical report generation subsystem 110 may determine that the terms, phrases, and/or utterances from the user-provided text are to be included in the updated text.

At step 1112, the updated auto-generated text may be stored in association with the content for inclusion in the draft medical report. For example, for auto-generated image 321 and/or image 331, the updated text may be used as auto-generated text 322 and/or auto-generated text 332, respectively, in draft medical report 300 based on the above provided example related to user-provided text being detected at time 230 (shown in FIG. 2). The auto-generated text may be presented in draft medical report 300, and the modifications to the auto-generated text based on the user-provided text may also be presented in draft medical report 300. Thus, the user may have the option to keep, ignore, or continue to edit the updated text in the draft medical report.

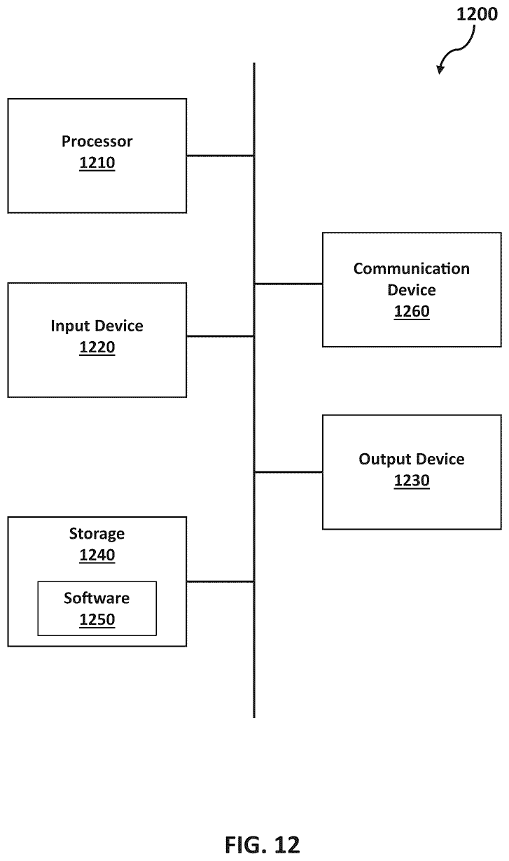

FIG. 12 illustrates an example computing system 1200, according to some aspects. Computing system 1200 may be used for performing any of the methods described herein, including method 800-1100 of FIGS. 8-11, respectively, and can be used for any of the systems described herein, including computing system 102 (and the subsystems included therein), medical device 120, client device 130, or other systems/devices described herein. Computing system 1200 can be a computer coupled to a network, which can be, for example, an operating room network or a hospital network. Computing system 1200 can be a client computer or a server. As shown in FIG. 12, computing system 1200 can be any suitable type of controller (including a microcontroller) or processor (including a microprocessor) based system, such as an embedded control system, personal computer, workstation, server, or handheld computing device (portable electronic device) such as a phone or tablet. The system can include, for example, one or more of processor 1210, input device 1220, output device 1230, storage 1240, or communication device 1260.

Input device 1220 can be any suitable device that provides input, such as a touch screen, keyboard or keypad, mouse, gesture recognition component of a virtual/augmented reality system, or voice-recognition device. Output device 1230 can be or include any suitable device that provides output, such as a touch screen, haptics device, virtual/augmented reality display, or speaker.

Storage 1240 can be any suitable device that provides storage, such as an electrical, magnetic, or optical memory including a RAM, cache, hard drive, removable storage disk, or other non-transitory computer readable medium. Communication device 1260 can include any suitable device capable of transmitting and receiving signals over a network, such as a network interface chip or device. The components of the computer can be coupled in any suitable manner, such as via a physical bus or wirelessly.

Software 1250, which can be stored in storage 1240 and executed by processor 1210, can include, for example, the programming that embodies the functionality of the present disclosure (e.g., as embodied in the devices as described above). For example, software 1250 can include one or more programs for performing one or more of the steps of the methods disclosed herein.

Software 1250 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 1240, that can contain or store programming for use by or in connection with an instruction execution system, apparatus, or device.

Software 1250 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic, or infrared wired or wireless propagation medium.

Computing system 1200 may be coupled to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Computing system 1200 can implement any operating system suitable for operating on the network. Software 1250 can be written in any suitable programming language, such as C, C++, C#, Java, or Python. In various examples, application software embodying the functionality of the present disclosure can be deployed in different configurations, such as in a client/server arrangement or through a Web browser as a Web-based application or Web service, for example.

As used throughout this application, the word “may” is used in a permissive sense (i.e., meaning having the potential to), rather than the mandatory sense (i.e., meaning must). The words “include”, “including”, and “includes” and the like mean including, but not limited to. As used throughout this application, the singular forms “a,” “an,” and “the” include plural referents unless the content explicitly indicates otherwise. Thus, for example, reference to “an element” or “a element” includes a combination of two or more elements, notwithstanding use of other terms and phrases for one or more elements, such as “one or more.” The term “or” is, unless indicated otherwise, non-exclusive, i.e., encompassing both “and” and “or.” Terms describing conditional relationships, e.g., “in response to X, Y,” “upon X, Y,” “if X, Y,” “when X, Y,” and the like, encompass causal relationships in which the antecedent is a necessary causal condition, the antecedent is a sufficient causal condition, or the antecedent is a contributory causal condition of the consequent, e.g., “state X occurs upon condition Y obtaining” is generic to “X occurs solely upon Y” and “X occurs upon Y and Z.” Such conditional relationships are not limited to consequences that instantly follow the antecedent obtaining, as some consequences may be delayed, and in conditional statements, antecedents are connected to their consequents, e.g., the antecedent is relevant to the likelihood of the consequent occurring. Statements in which a plurality of attributes or functions are mapped to a plurality of objects (e.g., one or more processors performing steps A, B, C, and D) encompasses both all such attributes or functions being mapped to all such objects and subsets of the attributes or functions being mapped to subsets of the attributes or functions (e.g., both all processors each performing steps A-D, and a case in which processor 1 performs step A, processor 2 performs step B and part of step C, and processor 3 performs part of step C and step D), unless otherwise indicated. Further, unless otherwise indicated, statements that one value or action is “based on” another condition or value encompass both instances in which the condition or value is the sole factor and instances in which the condition or value is one factor among a plurality of factors. Unless otherwise indicated, statements that “each” instance of some collection have some property should not be read to exclude cases where some otherwise identical or similar members of a larger collection do not have the property, i.e., each does not necessarily mean each and every. Limitations as to sequence of recited steps should not be read into the claims unless explicitly specified, e.g., with explicit language like “after performing X, performing Y,” in contrast to statements that might be improperly argued to imply sequence limitations, like “performing X on items, performing Y on the X'ed items,” used for purposes of making claims more readable rather than specifying sequence. Statements referring to “at least Z of A, B, and C,” and the like (e.g., “at least Z of A, B, or C”), refer to at least Z of the listed categories (A, B, and C) and do not require at least Z units in each category. Unless specifically stated otherwise, as apparent from the discussion, it is appreciated that throughout this specification discussions utilizing terms such as “processing,” “computing,” “calculating,” “determining” or the like refer to actions or processes of a specific apparatus, such as a special purpose computer or a similar special purpose electronic processing/computing device. Features described with reference to geometric constructs, like “parallel,” “perpendicular/orthogonal,” “square”, “cylindrical,” and the like, should be construed as encompassing items that substantially embody the properties of the geometric construct, e.g., reference to “parallel” surfaces encompasses substantially parallel surfaces. The permitted range of deviation from Platonic ideals of these geometric constructs is to be determined with reference to ranges in the specification, and where such ranges are not stated, with reference to industry norms in the field of use, and where such ranges are not defined, with reference to industry norms in the field of manufacturing of the designated feature, and where such ranges are not defined, features substantially embodying a geometric construct should be construed to include those features within 15% of the defining attributes of that geometric construct. The terms “first”, “second”, “third,” “given” and so on, if used in the claims, are used to distinguish or otherwise identify, and not to show a sequential or numerical limitation. As is the case in ordinary usage in the field, data structures and formats described with reference to uses salient to a human need not be presented in a human-intelligible format to constitute the described data structure or format, e.g., text need not be rendered or even encoded in Unicode or ASCII to constitute text; images, maps, and data-visualizations need not be displayed or decoded to constitute images, maps, and data-visualizations, respectively; speech, music, and other audio need not be emitted through a speaker or decoded to constitute speech, music, or other audio, respectively.

The foregoing description, for the purpose of explanation, has been described with reference to specific aspects. However, the illustrative discussions above are not intended to be exhaustive or to limit the invention to the precise forms disclosed. Many modifications and variations are possible in view of the above teachings. The aspects were chosen and described in order to best explain the principles of the techniques and their practical applications. Others skilled in the art are thereby enabled to best utilize the techniques and various aspects with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

1. A computer-implemented method, comprising:
generating a draft medical report comprising auto-generated content describing a medical procedure, wherein the auto-generated content comprises one or more auto-generated images that have been selected based on medical report criteria;
displaying the draft medical report comprising the one or more auto-generated images;
receiving a user selection of at least one of the one or more auto-generated images; and
updating the draft medical report based on the user selection of the at least one of the one or more auto-generated images.

2. The method of claim 1, further comprising:
selecting a medical profile of a user associated with the medical procedure, the medical profile comprising the medical report criteria.

3. The method of claim 1, further comprising:
identifying one or more time windows associated with the medical procedure; and
capturing an image during at least one of the one or more time windows, wherein the one or more auto-generated images comprise the captured image.

4. The method of claim 1, further comprising:
obtaining a medical profile of a user associated with the medical procedure; and
identifying one or more medical report preferences of the user based on the medical profile, the one or more medical report preferences indicating time windows of the medical procedure during which the user prefers to capture images, wherein the one or more auto-generated images comprise at least some of the captured images.

5. The method of claim 1, further comprising:
obtaining auto-generated text describing each of the one or more auto-generated images, wherein the auto-generated content comprises the auto-generated text, and wherein the draft medical report comprises the one or more auto-generated images and the auto-generated text corresponding to each of the one or more auto-generated images.

6. The method of claim 1, further comprising:
displaying the updated draft medical report comprising at least some of the auto-generated content and the at least one of the one or more auto-generated images.

7. The method of claim 1, wherein updating the draft medical report comprises:
adding the at least one of the one or more auto-generated images to the draft medical report to obtain the updated draft medical report.

8. The method of claim 1, further comprising:
determining one or more medical events associated with the medical procedure based on prior performances of the medical procedure; and
generating a medical profile of a user associated with the medical procedure, the medical profile storing data indicating the one or more medical events.

9. The method of claim 8, further comprising:
detecting, within a video of the medical procedure, at least some of the one or more medical events; and
selecting one or more images depicting the at least some of the one or more medical events, wherein the auto-generated content comprises at least some of the one or more images.

10. The method of claim 1, further comprising:
training a machine learning model to identify one or more image descriptors associated with phases of the medical procedure; and
capturing, from video of the medical procedure, one or more images corresponding to the phases of the medical procedure, the auto-generated content comprising at least some of the one or more captured images.

11. The method of claim 10, wherein the one or more image descriptors comprise at least one of objects, environmental factors, or contextual information associated with the phases of the medical procedure.

12. The method of claim 11, further comprising:
generating training data comprising images that were captured during prior performances of the medical procedure for training the machine learning model; and
storing at least one of the trained machine learning model or the training data in association with a medical profile of a user that performed the medical procedure.

13. The method of claim 11, further comprising:
detecting, within video of the medical procedure, using the trained machine learning model, at least one of the one or more image descriptors; and
selecting one or more images from the video of the medical procedure depicting the at least one of the one or more image descriptors, the auto-generated content comprising at least some of the one or more selected images.

14. The method of claim 1, further comprising:
determining time windows associated with phases of the medical procedure; and
detecting an image captured at a time different than the time windows, wherein the one or more auto-generated images comprise the detected image.

15. The method of claim 1, further comprising:
associating audio captured during the medical procedure with an image captured during a time window associated with a phase of the medical procedure.

16. The method of claim 1, further comprising:
receiving user-provided text;
merging the user-provided text with auto-generated text associated with the captured image, wherein the draft medical report comprises the captured image and the merged text.

17. The method of claim 1, wherein generating the draft medical report comprises:
determining, based on a medical profile of a user associated with the medical procedure, one or more medical report preferences of the user; and
creating the draft medical report based on the one or more medical report preferences.

18. The method of claim 1, further comprising:
generating, using a machine learning model, auto-generated text for the one or more auto-generated images, wherein updating the draft medical report comprises:
adding the auto-generated text associated with at least one of the one or more auto-generated images to the updated draft medical report.

19. A system, comprising:
one or more processors programmed to perform a method comprising:
generating a draft medical report comprising auto-generated content describing a medical procedure, wherein the auto-generated content comprises one or more auto-generated images that have been selected based on medical report criteria;
displaying the draft medical report comprising the one or more auto-generated images;
receiving a user selection of at least one of the one or more auto-generated images; and
updating the draft medical report based on the user selection of the at least one of the one or more auto-generated images.

20. A non-transitory computer-readable medium storing computer program instructions that, when executed, effectuate a method comprising:
generating a draft medical report comprising auto-generated content describing a medical procedure, wherein the auto-generated content comprises one or more auto-generated images that have been selected based on medical report criteria;
displaying the draft medical report comprising the one or more auto-generated images;
receiving a user selection of at least one of the one or more auto-generated images; and
updating the draft medical report based on the user selection of the at least one of the one or more auto-generated images.
